(19) United States
(12) Patent Application Publication
(10) Pub. No.: US 2012/0136416 A1
(43) Pub. Date: May 31, 2012

(54) Caged Neuropeptides: modulation and measurement of synapse and signaling activity; and methods for drug evaluation, pharmaceuticals preparation and treatment

(75) Inventors: Bernardo L. Sabatini, Newton, MA (US); Matthew R. Banghart, Cambridge, MA (US)

(73) Assignee: PRESIDENT AND FELLOWS OF HARVARD COLLEGE, Cambridge, MA (US)

(21) Appl. No.: 13/299,613

(22) Filed: Nov. 18, 2011

Related U.S. Application Data
(60) Provisional application No. 61/415,730, filed on Nov. 19, 2010.

(51) Int. Cl. A61N 5/06; C07K 7/06; C07K 5/107; C07K 14/595; G01N 33/566; C07K 2/00

(52) U.S. Cl. 607/92; 435/7.21; 530/300; 530/330; 530/324; 530/328

(57) ABSTRACT

A neuropeptide is caged and delivered to a synaptic site, then photolytically activated to enable precise temporal and spatial temporal investigation of localized signaling activity or synaptic responses. Caged molecules, delivery and activation systems clarify the kinetics and activity of neuropeptide-mediated signaling to identify agonists and permit focused drug evaluation for specific diseases and conditions. The caged peptides may also provide light-mediated treatment therapies for localized relief of pain or nerve-related conditions, and serve as a screening or measurement tool for potential agonists and antagonists. A kit includes the caged molecule and may further include a reporter for indicating response to the activated neuropeptide.

### RELATED APPLICATION

This application is related to and claims the benefit of provisional application 61/415,730 of inventors Sabatini and Banghart, filed Nov. 19, 2010. The full text, drawings and other disclosure of that application are hereby incorporated herein by reference in their entirety, including its Appendices A-E which are discussed in the text below.

### GOVERNMENT SUPPORT CLAUSE

The invention herein was made with government support from the National Institute of Mental Health, grant 1R01MH085498-01. The government has certain rights in the invention.

### TECHNICAL FIELD

The present invention relates to caged neuropeptide compositions and methods for identifying and measuring synaptic signaling mechanisms and activity; and to methods for screening, evaluating and determining effects of and/or for designing effective pharmaceutical materials and preparations to modulate or control synaptic activity and neuronally-mediated processes, as well as to research tools for detecting, quantifying and normalizing peptide activity; for accurate time- and/or spatial measurements of synaptic activity and locally operative synaptic signalling mechanisms to identify effective pharmacological agents for treatment of pain, organic or traumatic dysfunction, and mental or other conditions involving transmission of neuronal signals.

### BACKGROUND

Neurons in the mammalian brain communicate via contact points, called synapses, at which chemical signals are exchanged. Synapses are functionally diverse. They include fast-signaling synapses that rely on the release and detection of amino acids or their derivates such as glutamate, glycine, and GABA; these synapses are in turn regulated by neuromodulatory synapses that signal through amino acid derivates such as dopamine, norepinephrine, and serotonin as well as the quaternary amine acetylcholine.

The molecular mechanisms of release of these neurotransmitters, and biochemical consequences of activation of their receptors, have been the focus of much study and investigation. As a result, their roles in the transmission and storage of information and in the regulation of neuronal circuits are relatively well-established. Furthermore, these signaling systems are known to be perturbed in many human neurological and psychiatric diseases including Parkinson's disease, schizophrenia, depression, and drug addiction.

A physiologically distinct and less understood system of synaptic signaling exists in parallel to and interwoven with these classical neurotransmitters. This is a system that signals through the release of short peptides that typically bind to and activate G-protein coupled receptors (GPCRs) to modulate cellular excitability and synaptic transmission. (Pennefather, J. N. et al. (2006) Life Sci 74, 1445-63; Snyder S. H. (2004) Neuropharmacology 47, 274-285; and Zeitzer, J. M. et al. (2006) Trends in Pharmacological Sciences 27, 368-374). The mechanisms by which these “neuropeptides” regulate circuit and animal behavior has been extensively studied in invertebrates such as Drosophila, Aplysia, crabs, and lobsters as well as in lower vertebrates such as the lamprey. (Nusbaum, M. P. et al. (2001) Trends in Neurosciences 24, 146-154; Joosse J. (1986) Prog Clin Biol Res 205, 13-32; Kandel E. et al. (1995) Science 268, 825-6; and Brocard F. et al. (2005) J Neurophysiol 93, 2127-2141). At the cellular level, while basic physiological responses to many neuropeptides have been described, the spatial and temporal precision of such signaling remain substantially unknown, and the picture may be confounded by the co-occurrence of other active peptides that may or may not participate in observed effects.

Neuropeptides are expressed and secreted throughout the mammalian brain, typically in combination with a fast neurotransmitter such as glutamate or GABA (Hokfelt, T. et al. (2000) Neuropharmacology 39, 1337-1356). Neuropeptides are packaged in vesicles and several neuropeptides are known to be released in an activity-dependent manner (Ludwig, M. et al. (2006) Nat Rev Neurosci 7, 126-136). Neuropeptide expression is often regulated by neuronal activity and many neurons are classified by their selective expression of different neuropeptides and neuropeptide receptors (Hokfelt, T. et al. (2000) Neuropharmacology 39, 1337-1356). Such regulated and heterogeneous expression of neuropeptides suggests a precise function in neuron-to-neuron signaling. Indeed, many aspects of synapse and cell function are modulated by neuropeptide-dependent activation of GPCRs (Strand, F. L. (1999) F. L. Strand, ed. (Cambridge, M A, The MIT Press); Tallent M. K. (2008) Results Probl Cell Differ 44, 177-200). At the behavioral level, neuropeptides have profound and complex neuromodulatory effects on brain function: they regulate social bonding (Insel, T. R. (2010) Neuron 65, 768-779), feeding (Morton, G. J. et al. (2006) Nature 443, 289-295), sleep (Adamantidis, A. et al. (2010) Front Mol Neurosci 2, 310), aversion (Knoll, A. T. et al. (2010) Brain Res 1314, 56-73) and reward (Le Merrer, J. et al. (2009) Physiol Rev 89, 1379-1412).

Studies into neuropeptide systems have been limited by a paucity of experimental tools. The conditions that trigger neuropeptide release from neurons are largely unknown and currently available methods of activating neuropeptide receptors in brain tissue prevent quantitative studies of their function. Although small molecule agonists for many neuropeptide receptors are available, many GPCRs exhibit functional selectivity such that they are incompletely or unnaturally activated by synthetic ligands (Urban, J. D. et al. (2007) J Pharmacol Exp Ther 320, 1-13). Furthermore, neuropeptides can bind and activate multiple receptor subtypes present on the same cell with similar affinities (Lupica, C. R. et al. (1992) Brain Res 593, 226-238; Svoboda, K. R. et al. (1999) J Neurosci 19, 85-95). Thus exogenous application of peptide ligands, rather than synthetic agonists, more accurately mimics endogenous peptidergic signaling. However, compared to traditional pharmacological agents, peptides are large, hydrophobic molecules and thus diffuse slowly within the brain. Direct peptide application in vivo and in brain slices by perfusion, pressure injection (Williams, J. T. et al. (1982) Nature 299, 74-77) or iontophoresis (Travagli, R. A. et al. (1995) Journal of Neurophysiology 74, 519-528) produces a slowly rising, prolonged and spatially imprecise presentation of the peptide. These methods offer poor control over the concentration of peptide delivered, largely limiting quantitative analysis to the effects of saturating doses for consistency (Duggan, A. W. et al. (1983) Pharmacol Rev 35, 219-2813). However, such doses can rapidly trigger receptor desensitization and internalization, which limits robustness and experimental throughput. Therefore, typical peptide delivery methods can only reveal slow and spatially imprecise neuropeptide actions, leaving the possibility of short-lived, local neuropeptide signaling unexplored.

In dissociated neurons, peptide signaling reaches full activation within several seconds of agonist exposure and deactivates within seconds of washout (Ingram, S. et al. (1997) Molecular Pharmacology 52, 136-143). However, in intact brain tissue, neuropeptide receptors are often found up to hundreds of microns from peptide release sites (Khachaturian, H. et al. (1985) Trends Neurosci 8, 111-119) suggesting that neuropeptides are capable of volume transmission. Indeed, strong evidence for this phenomenon has been generated in the spinal cord (Duggan, A. W. (2000) Prog Brain Res 125, 369-380). The spatiotemporal extent of neuropeptide signaling will be determined by the poorly understood interactions of rapid GPCR signaling downstream of ligand binding, slow peptide diffusion and the action of extracellular peptidases, so the limits of neuropeptide signaling in the brain remain undefined.

Neuropeptides and neuropeptide receptors are known to strongly modulate mammalian behavior, and deficits in neuropeptide signaling contribute to the pathogenesis of some human diseases. A number of drugs that target peptide receptors are used therapeutically for the treatment of disease; some of these drugs also see widespread abuse for recreational use and in addictive behaviors.

However, despite their importance in human biology and disease, the functions of most neuropeptides and neuropeptide receptors within the mammalian brain remain unknown, and the effects of neuropeptide release on cellular and synaptic physiology remain unclear. Fundamental biological questions remain about the spatial and temporal precision with which neuropeptides act, and about the signals that trigger their release. One neuropeptide may operate as a neurotransmitter in different synaptic contexts to trigger release of another neuropeptide, to inhibit release at a presynaptic level, or to inhibit an uptake. The relatively poor understanding of the roles of neuropeptide signaling in regulating synapses, neurons, and brain circuits results largely from a basic paucity of pharmacological tools for initiating and observing, or measuring, synaptic events and parameters.

Among the many peptides that act within the brain to regulate complex mammalian behaviors, may be included the Orexins (aka Hypocretins), which regulate sleep and appetite; Leptin, Neuropeptide Y, and Ghrelin, which regulate appetite, feeding, and energy metabolism; and Vasopressin and Oxytocin, which influence maternal behavior and bonding in mating pairs. Furthermore, deficiencies of Leptin, Orexin A, and Vasopressin directly cause human disease, and perturbations of endogenous opioids and Substance P have been proposed to contribute to psychiatric illness. Yet, with the exception of the Orexins, the rapid effects of these peptides on classical neurotransmission are largely unknown. Because of our limited knowledge of the roles of neuropeptides in regulating CNS neurons, it is difficult to predict how perturbations of these systems may disrupt synapse, cell, and circuit function in the brain. A primary obstacle preventing better understanding of the function of neuropeptide signaling is the unavailability of small-molecule, high-specificity agonists for the vast majority of neuropeptide receptors. A number of small metabolically active molecules such as glutamate and GABA (γ amino butyric acid) have been caged and released with high resolution light activation to enable well-correlated observations at the cellular level (Ellis-Davies, G. C. R. (2007) Nature Methods 4, 619-628), but it remains the case that the only way to activate most specific classes of neuropeptide receptors in brain tissue is to add the corresponding neuropeptide ligand to a perfusing solution.

Since peptides are sticky and diffuse slowly through brain tissue, this mode of application results in a slow-rising, prolonged, and spatially uniform presentation of the peptide, potentially affecting many populations of cells non-specifically, and potentially initiating or inhibiting multiple different activities in different cells, so that individual actions would be masked and not discernible. Administration by perfusion in a clinic or laboratory does not mimic the time course or distribution of natural peptide signaling in the brain, and can only reveal slow and spatially imprecise actions of neuropeptides. Also, prolonged exposure of G-protein coupled receptors to their ligands generally leads to desensitization and internalization of the receptors, effectively preventing a clear analysis of signaling via neuropeptide receptors in their native states. This observational limitation has resulted in the generally accepted and currently experimentally supported view of neuropeptides—as acting slowly and without spatial precision, and as being generally weak modulators of neuronal function. However such a view, reflecting present poor understanding based on inadequate tools for observing the roles of neuropeptide signaling in regulating synapses, neurons and brain circuits, is a scientifically unsatisfying foundation for the many pharmacological treatments of nerve-related or mental conditions that are nonetheless now being applied in the field.

It would therefore be useful to develop methods for precise or localized delivery of a small active neuropeptide.

It is also desirable to develop methods for observing and measuring the signaling activity of a synapse and its response to a locally released neuropeptide.

It is also desirable to develop methods for experimentally evaluating the effect on a synapse of a drug so as to screen agonists or potentially therapeutic preparations.

### SUMMARY

These and other inventive goals are achieved in accordance with aspects of the invention by caging a neuropeptide, delivering the cage material to a site, and selectively illuminating the site to photolytically activate the neuropeptide, thus overcoming the above-noted technical limitations and gaining insight into the spatiotemporal dynamics of peptidergic signaling. The photoactivatable neuropeptides can be applied to brain tissue at high concentrations in an inert form, and these molecules can be rapidly photolyzed to trigger release of the endogenous neuropeptide with high temporal and spatial precision. Initial experimental undertakings are reported below for opioid neuropeptides, since these short peptides and their receptors are known to regulate pain sensation (Scherrer, G. et al. (2009) Cell 137, 1148-1159), behavioral reinforcement (Le Merrer, J. et al. (2009) Physiol Rev 89, 1379-1412) and addiction (Gerrits, M. A. et al. (2003) Eur Neuropsychopharmacol 13, 424-434). Opioid peptides and their receptors are prominent in many brain regions including hippocampus, cerebellum, striatum, amygdala and the locus coeruleus (Khachaturian, H. et al. (1985) Trends Neurosci 8, 111-119; Mansour, A. et al. (1994) J Comp Neurol 350, 412-438). The opioid receptor family consists of three classically-recognized receptors: mu, delta and kappa. These are activated with differential affinity by the endogenous opioid peptides enkephalin and dynorphin and all couple to Gα_{i/o} such that their activation typically inhibits electrical excitability and neurotransmitter release via the opening of K⁺ channels and inhibition of voltage-sensitive calcium channels (Wagner, J. J. et al. (1995) Neuropharmacology of Endogenous Opioid Peptides (New York, Raven Press, Ltd.)).

To enable rapid, spatially delimited delivery of opioid peptides in neural tissue, applicants have developed ‘caged’ L-Enk and Dyn-8 peptides that can be released by exposure to ultraviolet (UV) light. These peptide analogues contain a photolabile chemical moiety in a position that attenuates activity at opioid receptors. Exposure to light causes the blocking group to detach, thereby releasing the peptide agonists. As photolysis occurs within microsecond kinetics, release can be initiated on the timescale of neurotransmission. Importantly, the concentration and spatial extent of released peptide can be regulated by varying laser power and the area of illumination. Experiments reported below characterize these caged neuropeptide research tools in brain slices, and define the spatiotemporal dynamics of opioid signaling with unprecedented resolution. By applying the caged neuropeptide and using targeted illumination to quickly and selectively convert it to active form at a defined locus, applicant is able to detect and quantify its activity and signaling roles in the brain or nervous system; clarify its role(s) in diseases and syndromes; and undertake better-directed and more effective therapies. The technology for the first time achieves controlled delivery of small neuropeptides to cellular processes such as single neurons, synaptic heads and dendrite spines. Proof-of-principle experiments were carried out with peptides that bind to opioid receptors, which were synthesized with a photo-cleavable caging group, applied in bulk and then light-activated as reported below to provide precise doses of the active neuropeptide at precise instants in time. A series of observations at one or more synaptically-connected positions then allows strong correlation with observed physiological effects, and characterization of baseline and response activity.

The neuropeptide or candidate neuropeptide agonist is linked to a chemical group forming a cage structure having a photolysable bond, wherein the cage structure blocks activity of the peptide, for example by creating steric hindrance to prevent the neuropeptide from interacting with a receptor. The cage is positioned at or near a receptor recognition subsequence of the neuropeptide. Examples are YGGFM (SEQ ID NO: 1) (Met-Enkephalin), YGGFL (SEQ ID NO: 2) (Leu-Enkephalin) for g and d opioid receprtors or the initial YGGFL (SEQ ID NO: 2) subsequence of the longer Dynorphin A neuropeptide for the κ-opioid receptor. In an embodiment of the invention, the cage may be a 2-nitrobenzyl caging group, or a dimethoxynitrobenzyl (DMNB) group, and may be applied to a side chain or to the backbone of the neuropeptide. For initial investigations to provide a strong activation pulse and detect resulting signaling activity, DMNB caging was selected as the caging agent for its greater sensitivity and fast photolysis. This choice of caging group, with ultraviolet uncaging stimuli, provides a sufficiently localized release to support exploration of single-synapse activation.

The caged peptide is applied to a tissue or a cell containing synapses or G protein coupled receptors in vivo or in vitro, and light energy is applied to break the cage, thereby releasing the active peptide and initiating its interaction with the receptor. The light energy may be applied at a well defined location (for example by a controlled and focused scanner, or by a locally-positioned fiber optic light pipe), and at a specific time and/or in a specific amount. By observing resulting effects one determines an activation threshold, or confirms an associated neuropeptide signaling mechanism as well as spatial and temporal relationship of the stimulus and response in a synapse. Because the quantity of activated neuropeptide will be proportional to the amount of light delivered, precise dosing and measurements are possible, allowing computational assessment of the uptake and response kinetics. Measurements of related effects such as K- or Ca-channel conductivity of the synapse, release of signaling molecules and other effects or processes initiated in the synaptic chain are thus precisely correlated with activation of the neuropeptide or candidate neuropeptide agonist. These secondary responses may play important roles in diverse physiological events such as secretion, axoplasmic flow, motility, contraction, enzymatic reactions and membrane permeability. Such elucidation of neuromodulatory responses has a direct bearing on known or presumed pharmacological activities and improvement of existing neuropeptide-based therapies.

An aspect of the invention herein provides a method for evaluating response to a small neuropeptide, wherein the response is a neuronal activity parameter such as an organic state or susceptibility, a chemically- or biologically-induced response, or other condition or response of signaling cells or synapses.

Another aspect of the invention is a method for separating, characterizing or measuring complex peptide/synapse interactions, and identifying specific responses and secondary effects.

Yet another aspect of the invention is the identification of suitable treatment drugs for conditions mediated by neuronal signaling, such as pain, motility, mood and mental activity or conditions.

Methods of the invention include applying a caged peptide to a cell, the caged peptide comprising a peptide having an affinity for a receptor on a signaling cell and having a cage bound to the peptide and operative to prevent binding to the receptor, wherein the cage is photodegradable by application of light; applying light energy at an initial time t₀ to free the peptide for interaction with the receptor; and performing a measurement of activity to evaluate the parameter.

Methods of the invention include the step of applying the caged neuropeptide to a locus of interest, such as a neuronal site, irradiating the caged neuropeptide to break the cage so that the neuropeptide becomes active, e.g., freed to interact with a signaling cell or synapse. The irradiation may be localized, e.g., at one or more cells to initiate activity in a synaptic signaling junction and thereby permit measurement of localized activity at a specified time or specific position, or enable time-resolved measurements of kinetics. Alternatively, the irradiation may be applied to a region of tissue to release a caged neuropeptide previously supplied to the region. This mode of application may support bulk kinetic measurements for research or diagnostic purposes, or may provide a controlled therapeutic treatment, such as pain relief, in the treated region. The radiation may also be controlled to shape a delivery profile of the time and amount of neuropeptide delivered to a site, similarly to what is now done with programmable pumps, or with iontophoretic and other medication delivery systems. Thus, for example, an implanted fiber may be positioned and actuated to treat an epileptic brain lesion, either with a pre-programmed sequence of pulses, or user-actuated on-demand illumination. In experimental protocols, the neuropeptide may be applied to and released in one region of a mammalian brain to affect a synaptic head, while a response is measured distally, in another region to characterize the induced signaling response.

For elucidating the strength and mechanism of action of a candidate neuropeptide, the methods allow precise and localized measurements of activity, thus enabling the evaluation of speed of action, relative strength or effectiveness of a formulation; improved correlation with and observation of related effects such as release or uptake of other signaling molecules. Effects such as the threshold concentrations for activation or for saturation of the target receptors, measurement of calcium or potassium channel potentials or currents, and efficacy of competitive or blocking agents may also be measured. Methods may be applied to elucidate cellular pathways and synaptic signaling mechanisms, to screen drug candidates, and to evaluate dosage, lifetime and other characteristics of potential treatment drugs and formulations. A kit may include a substantially pure caged neuropeptide for controlled release to measure a response.

Therapeutic methods may include treatment of wounds or disease states to block or inhibit pain, or to modulate synaptically-mediated tissue activity; initiation of local interactions to provide diagnostic tests of neurological conditions, and other medical and therapeutic applications.

### BRIEF DESCRIPTION OF THE DRAWINGS

These and other aspects of the invention will be understood from the description below (including discussion of drawings of Appendices A, B, C, D and E of the above-referenced provisional application 61/415,730) and the claims appended hereto, taken together with the Figures and illustrative examples herein, wherein:

FIG. 1 schematically illustrates caging and photolytic release of a bioactive molecule using a nitrobenzyl caging group;

FIG. 2 illustrates several approaches for caging peptides for use in the invention;

FIGS. 3A, 3B and 3C illustrate several caged Enkephalin and Dynorphin analogues, caged SP analogues, and caged CCK analogues;

FIG. 4 illustrates pharmacological validation of the photoactivable caging of three different Leu-Enkephalin analogues.

FIG. 5 A-D show CYLE and CYD8, caged L-Enk and Dyn-8 analogues, amino acid sequences and caging structure, dose-response curves normalized to endogenous peptide agonists at delta, mu and kappa opioid receptors;

FIG. 6 shows outward current signal traces in neurons of the LC activated by photorelease of L-Enk, Dodt contrast images of brain stem slices, silencing of spontaneous action potentials, outward currents in a LC neuron, peak and average currents and overlays of repetitively sample responses to uncaging stimuli;

FIG. 7 shows Graded activation of outward currents in LC neurons evoked by photorelease of L-Enk at different power levels and different uncaging areas, showing current and voltage responses;

FIG. 8 shows activation of K⁺ currents by L-Enk, measurement protocol and baseline current, and comparison with modeled K⁺ reversal potential;

FIG. 9 shows effect of peptidase inhibitors on light-evoked current and charge transfer for different uncaging beam areas;

FIG. 10 shows activation kinetics as function of proximity of stimulus to soma;

FIG. 11 shows chromatograms of natural neuropeptides and corresponding caged and photoreleased material confirming clean photoconversion;

FIG. 12 shows dose response curves of caged and native neuropeptides at kappa and delta opioid receptors;

FIG. 13 is a Table summarizing dose-response data;

FIG. 14 shows an isomer of CYLE and response to uncaging stimulus of FIG. 6 in brain slices of LC;

FIG. 15 shows lack of effect of the gap junction inhibitor carbenoxolone on light-evoked current responses to uncaging stimuli as used in FIG. 7; and

FIG. 16 shows spatial profile of L-Enk responses to different size uncaging spots of focused light.

### DETAILED DESCRIPTION

### Development of Caged Opioid Neuropeptides

Neuropeptides have profound and complex neuromodulatory effects on neuronal function in the mammalian nervous system. By activating G protein coupled receptors (GPCRs), neuropeptides bidirectionally modulate cellular excitability and synaptic transmission. However, the spatiotemporal precision of their actions remain undefined. Neuropeptides are generally believed to function through volume transmission although close association between release sites and receptors is frequently observed, suggesting a role for spatially restricted, synapse-specific signaling. Furthermore, the same receptors can often be found in dendrites, somata and axons of a cell, but each subset appears to mediate a different physiological response and might be activated independently. Currently available methods of applying neuropeptides to brain tissue limit quantitative studies of their activity. Compared to traditional pharmacological agents, peptides are large, hydrophobic molecules and thus diffuse very slowly. Application in vivo and in brain slices by perfusion, pressure injection or iontophoresis produces a slowly rising, prolonged and spatially imprecise presentation of the peptide with poor control over concentration. Such delivery methods are unlikely to mimic the natural time-course of peptide signaling in the brain and can only reveal slow and spatially imprecise actions.

To enable rapid, spatially delimited delivery of neuropeptides in neural tissue, Applicants developed ‘caged’ neuropeptides that can be released, that is rendered active, by exposure to light. These peptide analogues contain a photolabile chemical moiety or blocking group in a position on the peptide that attenuates activity at the target receptor. Exposure to light causes the blocking group to detach, thereby releasing the endogenous neuropeptide. Because brain tissue can be pre-equilibrated with the inactive caged peptide and light can be delivered with submicron precision, neuropeptides can be released onto specific sub-cellular compartments independently. Photolysis may occur within microseconds, so release can be controlled on the timescale of vesicular fusion. Importantly, the concentration of released peptide can also be quantitatively regulated by varying light intensity and/or flash duration.

To enable delivery of neuropeptides on the scale of single synapses with millisecond kinetics, applicants have caged neuropeptides to deactivate their recognition domain with a photo-cleavable caging group. Initial development reported herein employs nitrobenzyl as a caging group on a terminal amino acid of all or a portion of the peptide binding recognition sequence, such as tyrosine of a Leu- or Met-Enkephalin, with a bond that is broken by light of an appropriate frequency. The nitrobenzyl caging system was chosen for its effectiveness, and the speed and efficiency of photolysis, which allows complete activation in under a millisecond with UV light. Because UV is highly absorbed, and therapeutic application to treat internal tissues by activating the peptide would require exposure of the material to light, this system as applied to therapeutic use may be limited to situations where the light can be applied to external tissue (such as skin), or be applied endoscopically (such as to the esophagus or to a surgically accessed site), or be applied via an optical fiber (e.g., a fiber implanted to a small neural lesion or other treatment site). However, applicants also contemplate that other suitable caging and photolysis systems may be employed or developed, including ones having much lower photoactivation energy, e.g., activated by longer wavelength light having lower energy but greater tissue-penetrating range. For such systems with lower energy photons, activation may be effected, for example, by two-photon activation at a localized site using synchronous scanning of infrared illumination to apply the necessary photolytic signal for release of the peptide at subsurface sites without causing radiation or thermal damage to intervening or surrounding tissue. Two-photon systems, previously used for enhanced resolution microscopy and fluorescent microscopy may thus be applied to cause precision delivery of active neuropeptide to a synaptic head or process.

### Proof of Principle

Applicants initially focused on the opioid family of neuropeptides, which play a central role in pain transmission, behavioral reinforcement and addiction. Opioid peptides and their receptors are prominent in many areas of the brain including the hippocampus, cerebellum, striatum, amygdala and the locus coeruleus (LC). Enkephalins were selected as particularly attractive targets due to their small size and established pharmacology. The initial five pages of Appendix A of the above-mentioned provisional patent application identify some opioids and known physiological or molecular responses associated with them. Below we discuss the caging approaches and several validation assays for confirming caging and restoration of activity for compiling baseline measurements, followed by descriptions of proof of principle experiments and representative measurement results and observations of synaptic activity.

Building on published structure-activity relationships, several candidate caged Leu-Enkephalin (L-Enk) analogues containing nitrobenzyl-derived chromophores were designed and several potential synthetic routes to each compound were developed. Synthesis was outsourced to companies specializing in small molecules and peptides, and generally the synthetic material was further purified by preparative HPLC to remove residual traces of photolysis products prior to analysis.

The following nomenclature is adopted herein for two caged materials: CYLE: Carboxy-nitrobenzyl tyrosine (Y) Leucine-Enkephalin; and NYLE: Nitrophenethyl tyrosine (Y) Leucine-Enkephalin.

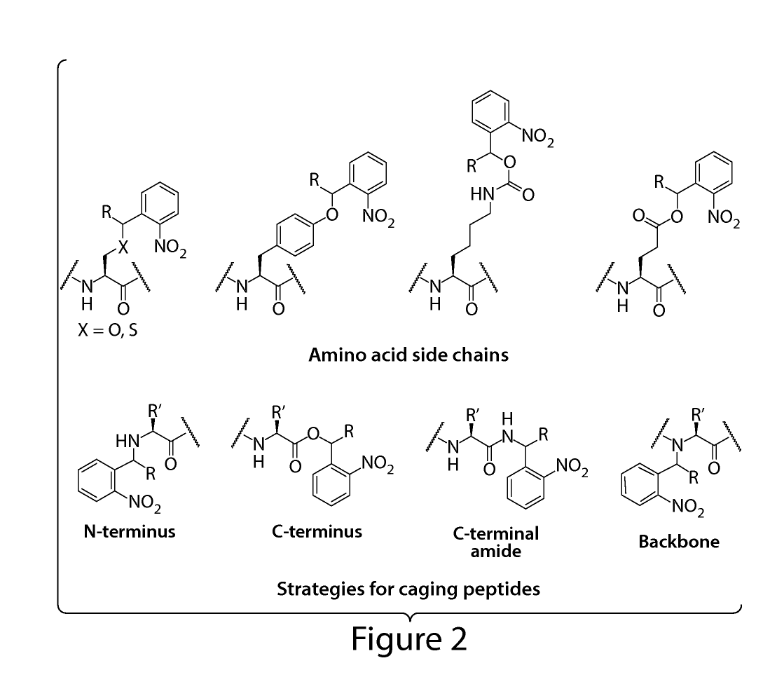

Several caging strategies were considered to inactivate bioactive peptides. For example, amino acid side chains that contain reactive atoms are modified with photo-removable protecting groups based on the nitrobenzyl-chromophore, as shown in FIG. 2. However, if these side chains are not crucial for peptide bioactivity, modifying them will not produce an inactive molecule. In this case, several other sites on peptides can be considered: the N-terminal amine, the c-terminal carboxylic acid, the c-terminal carbamide (which is present in many neuropeptides), or even the backbone amide nitrogen atoms.

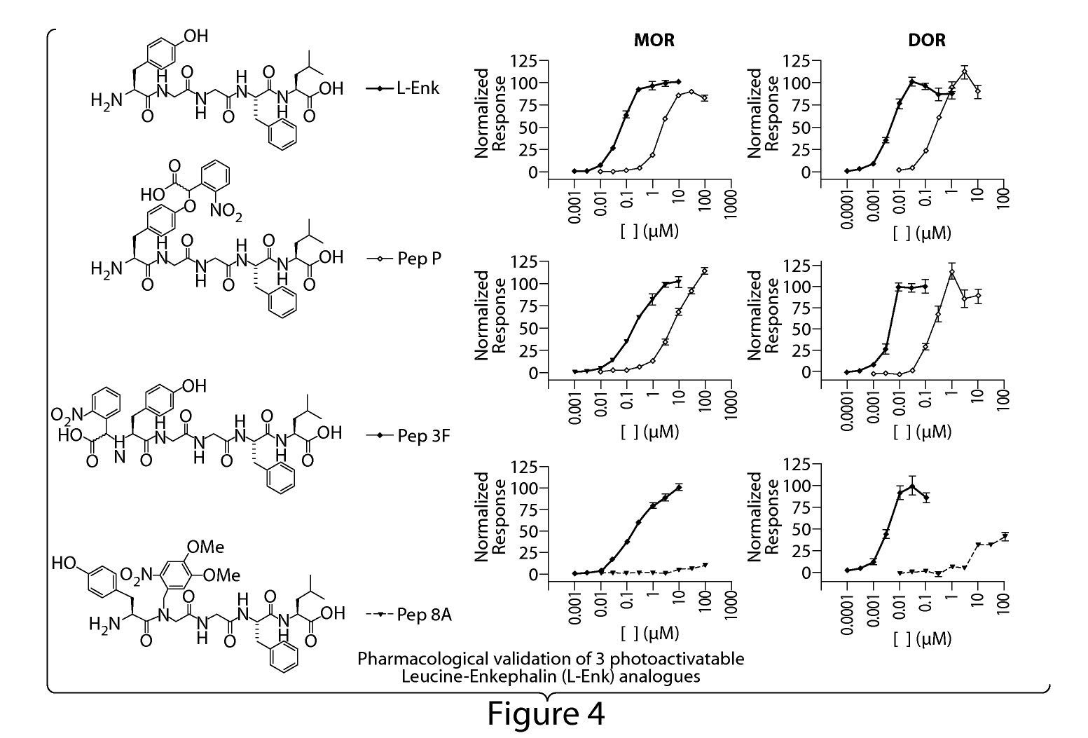

Several caged L-Enk analogues were designed and obtained from Peptech Corp. Caging the tyrosine side-chain was taken as a starting point because it is well established that the tyrosine phenol (OH) is essential for potent activity at opioid receptors. Mutating the tyrosine to phenylalanine reduces the activity by ˜100 fold, as does methylating it to form a methyl ether. Thus the caged analogues CYLE (Pep P in FIG. 4) and NYLE were designed and considered to be the most likely to work. (see the aforesaid Appendix E) Indeed, CYLE (Pep P) exhibits ˜100 fold reductions in potency at both Mu and Delta opioid receptors (MOR and DOR), consistent with the established role for this phenol group. Furthermore, the carboxynitrobenzyl modified tyrosine component has been shown by Sreekumar to exhibit optimal photolysis parameters such as high quantum yield (>20%) and fast kinetics such that photolysis is complete within several microseconds following the light flash, which is faster than any biological processes under study.

However, the N-terminus overall has been shown to be involved in binding to the receptor. Alkylating the N-terminal amine leads to antagonism, and alkylation with a large group such as nitrobenzyl has not yet been explored. Thus analogue Pep 3F (FIG. 4) was also obtained and was surprisingly found to exhibit similar activity to CYLE (PepP). However, it is also known that direct uncaging of amines that are alkylated with nitrobenzyl groups proceeds inefficiently (quantum yield of less than 0.1%) and very slowly (seconds) in aqueous solution. Therefore, this molecule was considered likely to have non-optimal photochemical properties that would limit its use in neural tissue.

Focusing on the essential pharmacological role of the N-terminal region of L-Enk, we also considered the back-bone caged compound Pep 8A shown in FIG. 4. Although this molecule appeared to have optimal pharmacological inactivity, it was found to be highly chemically unstable in aqueous solution, decomposing in several hours to yet un-identified by products. It is thus not useful in biological experiments.

Dose-response relationships are determined for each analogue and compared to L-Enk using a modified secreted alkaline phosphatase assay that enables Gi-coupled receptors to signal through the Gs pathway, using a live-cell assay developed by Dr. Steven Liberles at Harvard Medical School. Mu, Delta or Kappa opioid receptors are preferably co-expressed in HEK cells with a chimeric G protein that enables Gi-coupled receptors to signal through the Gs pathway. This leads to expression of a cAMP-dependent reporter gene as a function of ligand-induced receptor activation. A variety pharmacological assays for opioid receptors are well-established (ie: the FLIPR), and may be used. The specific assay employed herein was a secreted alkaline phosphatase (SEAP) assay technique (see schematic drawings in the aforesaid Appendix D and the Experimental Procedures section, infra), a live-cell assay in which a gene encoding the opioid receptor of interest (Mu, Delta, Kappa) is transfected into HEK 293 cells that stably express a chimeric G protein along with a reporter construct. This chimeric G protein is composed of regions of the Gs protein and Gi protein. The Gi protein element interacts with the naturally Gi-coupled Mu, Delta and Kappa receptors. The Gs protein element causes activation of the receptor to activate adenylate cyclase. This leads to transcription of the reporter construct according to the SEAP shown in Appendix D. The reporter construct is a secreted alkaline phosphatase (SEAP) which is secreted from the cells and accumulates in the extracellular medium as a function of receptor activation. The extracellular medium is treated with the pro-fluorescent compound methylumbelliferone phosphate (MUP), which is dephosphorylated by the SEAP protein to produce the fluorescent compound methylumbelliferone. The fluorescence intensity of methylumbelliferone is proportional to the degree of receptor activation. By plating these cells in 96-well plates, dose-response curves for the native and caged ligands (L-Enk and CYLE/NYLE) are generated conveniently with medium throughput.

Several analogues were identified that exhibit >100 fold reductions in potency with respect to L-Enk at both Mu and Delta receptors. Consistent with the selectivity of L-Enk, activity at Kappa receptors is absent, as was any indications of antagonism. For each analogue, photolytic conversion to L-Enk is studied using UV/VIS, HPLC and mass spectrometry.

Preliminary electrophysiological evaluation was carried out in transverse hippocampal slices from P15-18 mice and focused on inhibitory neurons, the predominant cells expressing Mu and Delta opioid receptors in the CA1 region. Initial neurophysiological evaluations were carried out in acute hippocampal slices from P24-30 mice. In the CA1 region of the hippocampus, GABA release from parvalubmin-expressing basket cells is selectively reduced by L-Enk through activation of Mu and Delta opioid receptors, leading to disinhibition and increased excitability in pyramidal cells. Extracellular field potential recordings reveal this as an increase in the population spike (pSpike) amplitude in response to Schaeffer collateral stimulation at 0.1 Hz. By adding the caged peptides in a bath solution Applicants could repetitively elicit rapid, transient increases in pSpike amplitude using brief (50-200 ms) UV light flashes, and these were blocked with the broad-spectrum opioid receptor antagonist naloxone. These experiments confirmed that photolysis in living tissue elicits a physiological response.

To more accurately evaluate the kinetics of the uncaging response, Applicants made whole cell recordings from locus coeruleus (LC) neurons in acute slices from P22-P28 rats. The LC is a homogenous nucleus of spontaneously firing noradrenergic cells that express a high density of Mu opioid receptors, activation of which is known to open GIRK channels and inhibit voltage gated calcium channels. In voltage clamp and current clamp recordings, 5 ms light flashes produced large outward currents and hyperpolarizations of the membrane potential, respectively. These uncaging responses always peaked within one second of the light flash but varied in magnitude and duration according to the size of the uncaging spot and distance from the cell body. Pharmacological manipulations allowed for quantitative investigations into the mechanisms of Enkephalin signaling in the LC. Several details are provided below for experimental results that are shown in the above-referenced Appendix B.

### Application of the Compound to Neural Tissue:

Because the caged molecule is hydrophobic in nature, aqueous solubility is limited at high concentrations for preparation of stock solutions. Therefore, mixtures of DMSO and water were used to prepare the stocks. The final concentration of DMSO in the brain slice preparations was reduced to less than 0.1% to avoid toxicity. A 10 millimolar stock was used to obtain the final concentration of 10 micromolar. The molecule was maintained as present in the entire bath solution which was re-circulated to minimize the amount of material used while providing a uniform concentration throughout the tissue.

### Photolysis in Acute Slices of Brain Tissue:

The details of the microscope configuration include: photolysis of caged neurotransmitters using 355 nm light (Wang S S H and Augusting G J 1996; Tang C-M 2006). However, in different drawings of the aforesaid Appendix B (drawings 4, 5, 6, 7, 8), slightly different spatial patterns of light were applied and also UV-blocking filters that were placed in the trans-illumination light path to prevent unintentional photolysis while establishing recordings from cells, which requires visualization of the cells and the recording electrode.

In drawing 4 of Appendix B, a “full field” uncaging strategy was used in which the collimated UV laser beam was focused on the back aperture of the microscope objective to produce a column of light through the sample which uniformly illuminates the entire field of view. This causes excitation of a large area uniformly and simultaneously in the x-y plane while the cell body was centered in the field of view. Thus the cell body was exposed to the photolyzed peptide, and the dendrites and axon were within the field of view to ensure a large electrophysiological response. The area of illumination corresponds to the red ring in drawing 7A of Appendix B.

In drawing 5 of Appendix B the collimated laser beam was directly entered into the microscope objective to create a small, 30 micron diameter spot of light at the focal point of the objective. This was centered on the cell body from which the recording was being made. The power was then adjusted using neutral density filters in the optical path. In this case the photolyzed peptide was being delivered only to the cell body. However, because the absolute power levels in this experiment were higher than in FIG. 4, even larger responses were obtained.

In drawing 6 of Appendix B the optics were similar to those described above for drawing 5 of Appendix B with the exception that the light spot was focused to a spot of only 2 microns in diameter to restrict the region of uncaging to minimal size and allow analyses of diffusion of the uncaged peptide from distant sites of release to the cell body.

In drawing 7 of Appendix B, the optical configuration was similar to that in drawing 4 with the exception that a field diaphragm (or aperture) was placed in a conjugate image plane of the laser path. This allowed the diameter of the collimated uncaging beam to be varied to areas smaller than the entire field of view.

Drawing 8 of Appendix B utilized the same configuration as drawing 4 and corresponds to the red ring in drawing 7A of Appendix B.

### Chemical Synthesis:

Caging with a photo-sensitive moiety (carboxy-nitrobenzyl tyrosine or CNB-Y) has been published (Sreekumar, R. et al. (1998) Methods Enzymol 291, 78-94). The pharmacological assay for opioid receptors was a secreted alkaline phosphatase (SEAP) assay.

### Application of the Compound to Neural Tissue:

Because the caged molecule is hydrophobic in nature, aqueous solubility is limited at high concentrations for preparation of stock solutions. Therefore, mixtures of DMSO and water were used to prepare the stocks. The final concentration of DMSO in the brain slice preparations was reduced to less than 0.1% to avoid toxicity. A 10 millimolar stock was used to obtain the final concentration of 10 micromolar. The molecule was maintained as present in the entire bath solution which was re-circulated to minimize the amount of material used while providing a uniform concentration throughout the tissue.

### Photolysis in Acute Slices of Brain Tissue:

The details of the microscope configuration include: photolysis of caged neurotransmitters using 355 nm light (Wang S S H and Augusting G J 1996; Tang C-M 2006). However, in different drawings shown in the above-referenced Appendix B (drawings 4, 5, 6, 7, 8), slightly different spatial patterns of light were applied and also UV-blocking filters that were placed in the trans-illumination light path to prevent unintentional photolysis while establishing recordings from cells, which requires visualization of the cells and the recording electrode. Dodt gradient microscopy was used in some experiments to visualize a process while positioning the uncaging spot and detection assay.

### Further Experimental Considerations and Procedures

### Peptide Synthesis, Purification and Stock Preparation

Custom chemical synthesis was carried out by Peptech Corporation (Burlington, Mass.) using standard Fmoc-based solid phase peptide synthesis. The carboxynitrobenzyl-modified tyrosine was prepared by modifying established protocols (Sreekumar, R. et al. (1998) Methods Enzymol 291, 78-94). After arrival, CYLE and CYD8 were typically handled under lighting filtered using Rosculux #312 Canary optical filter paper to remove any traces of UV light that could lead to unintentional photolysis. It was essential to further purify the synthetic material by semi-preparative reverse-phase high pressure liquid chromatography (RP-HPLC, Agilent) to remove contaminating photolysis products, which typically included ˜1% L-Enk or Dyn-8. Crucially, the UV (and VIS) lamps on the detector were turned off during the purification to prevent photolysis of the purified material. Purified, solid material was dissolved in 50% DMSO/water and diluted to a concentration of 10 mM. Stock concentrations were determined by UV/VIS spectroscopy using an extinction coefficient of 1,000 M⁻¹cm⁻¹ at 355 nm for the carboxynitrobenzyl-modified tyrosine (Sreekumar, R. et al. (1998) Methods Enzymol 291, 78-94).

### Photolysis in Solution

A 100 μl sample of either 1 mM CYLE or CYD8 in phosphate buffered saline, pH 7.2, contained in a quartz cuvette was placed for 30 s in the path a 100 KHz pulsed q-switched UV laser (DPSS, Santa Clara, Calif.) producing ˜800 mW of 354.7 nm light. The samples were then analyzed by RP-HPLC (Agilent) on a 4.6 mm×150 mm Zorbax SB-C8 column (5 μm particle size) in H₂O, MeCN, 0.1% TFA at 1 ml/min. L-Enk and CYLE were resolved by a gradient from 30% to 70% MeCN over 10 min Dyn-8 and CYD8 were resolved by a gradient from 25% to 70% MeCN over 10 min. Samples were monitored at 210 nm, 280 nm and 350 nm. Non-photolyzed CYLE, CYD8, L-Enk and Dyn-8 samples were prepared and run under the same conditions. Although addition of the parent peptides to photolyzed samples increased the product peaks as expected, further analysis by mass spectrometry confirmed photolytic conversion of CYLE to L-Enk and CYD8 to Dyn-8 (data not shown).

### Cell Culture and SEAP Assay

HEK293 cells stably transfected with a chimeric Gₛ-Gᵢ protein (Liberles, S. D. et al. A High Throughput Assay for GPCR Ligands Using G(alpha)s Chimeras. Submitted; Liberles, S. D. et al. (2006) Nature 442, 645-650) were grown in DMEM (Invitrogen) containing 5% FBS (Invitrogen) and 500 μg/ml G-418 (Invitrogen) and maintained at 37° C. in an atmosphere of 5% CO₂. Cells were plated in 96-well plates at 50,000 cells/well and co-transfected with the GPCR and reporter plasmid using Lipofectamine® (Invitrogen) and PLUS® reagent (Invitrogen). The transfection media was replaced with ligand-containing DMEM (200 μl/well) and cells were incubated for 36-48 hrs 37° C./5% CO₂. Care was taken to avoid exposure to bright, non-filtered light. Each plate was then wrapped in plastic wrap and incubated at 68° C. for 2 hrs to heat-inactivate native phosphatases. After transferring 100 μl aliquots from each well to a fresh 96-well plate, 100 μl of an aqueous buffer containing 2 M diethanolamine bicarbonate and 1.2 mM methylumbelliferone phosphate, pH 10.0, was added to each well. Plates were imaged using a Perkin Elmer Envision plate reader using optical settings for methylumbelliferone fluorescence, taking care to image all conditions to be compared on the same day for a single receptor at the same time-point after MUP addition. In every individual experiment, the non-caged parent ligand (L-Enk or Dyn-8) was evaluated alongside the caged compound (CYLE or CYD8) at the same receptor and used to normalize data across trials. cDNAs encoding human delta (OPRD1) and human kappa opioid receptors (OPRK1) contained an N-terminal 3×HA epitope tag (Missouri S&T cDNA Resource center, www.cdna.org) and the murine mu opioid receptor (OPRM1) contained an N-terminal FLAG epitope tag. Data shown reflect the average of 6 replicates run in parallel for each condition.

### Animal Handling and Slice Preparation

Animals were handled according to protocols that were approved by the university's Standing Committee on Animal Care and are in accordance with Federal guidelines. Post-natal day 22-29 Long-Evans rats were anesthetized by inhalation of isoflurane and cardiac perfused with ice-cold ACSF containing (in mM): 127 NaCl, 2.5 KCl, 25 NaHCO₃, 1.25 NaH₂PO₄, 2.0 CaCl₂, 1.0 MgCl₂, and 25 glucose, equilibrated with 95% O₂/5% CO₂, osmolarity 307. Horizontal slices of locus coeruleus were prepared in a cold choline-based artificial cerebrospinal fluid (choline-ACSF) containing (in mM): 25 NaHCO₃, 1.25 NaH₂PO₄, 2.5 KCl, 7 MgCl₂, 25 glucose, 1 CaCl₂, 110 choline chloride, 11.6 ascorbic acid, and 3.1 pyruvic acid, and equilibrated with 95% O₂/5% CO₂. Slices of 260 μm thickness were cut with a Leica VT1000s (Leica Instruments, Nussloch, Germany) and transferred to a holding chamber containing ACSF. Slices were incubated at 32° C. for 30-45 min and then left at room temperature (20-22° C.) until recordings were performed.

### Electrophysiology

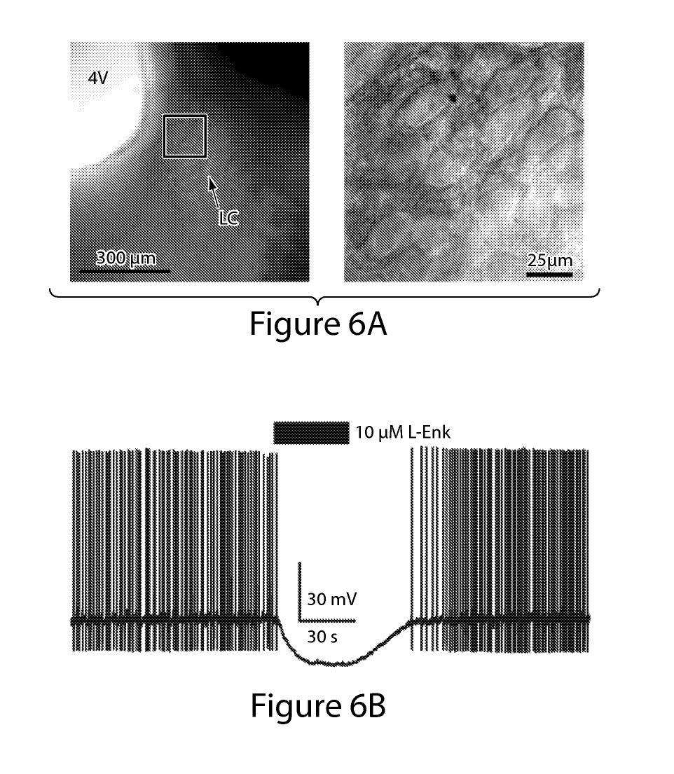

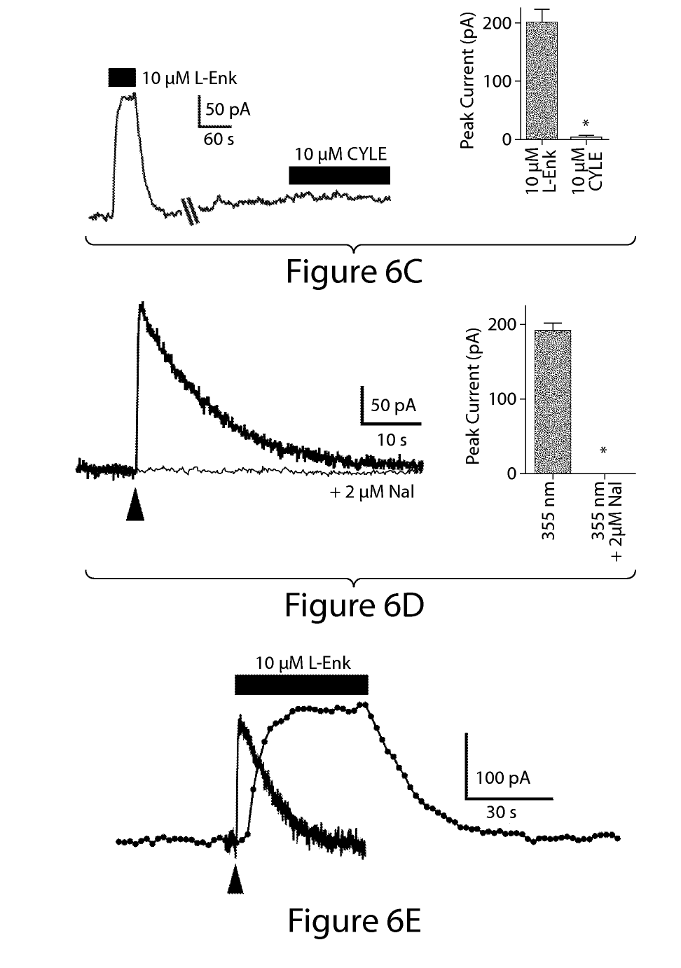

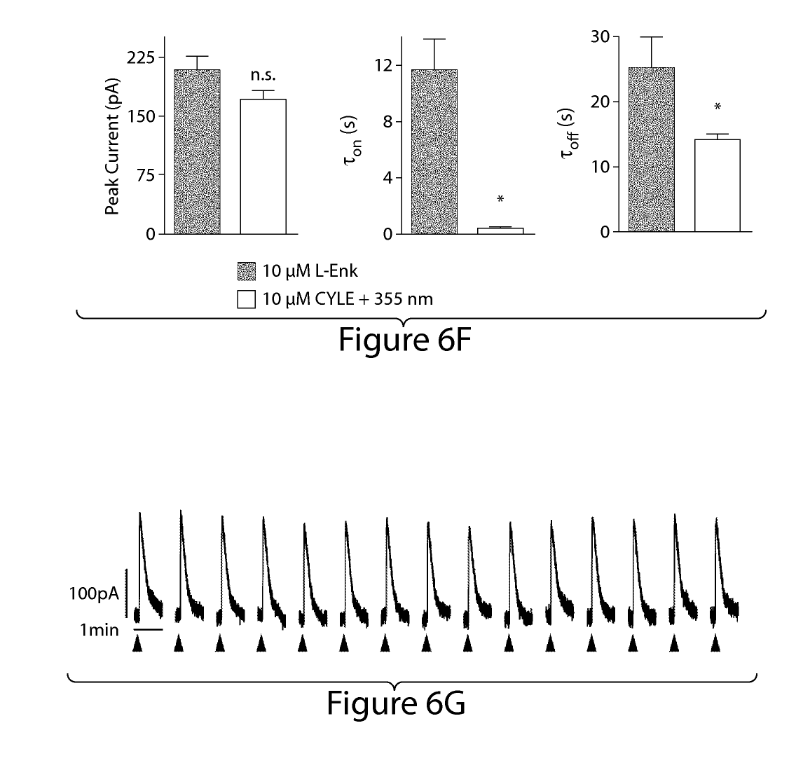

All recordings were, performed within 5 hours of slice cutting in a submerged slice chamber perfused with ACSF warmed to 32° C. and equilibrated with 95% O₂/5% CO₂. Whole-cell recordings were made from LC neurons visualized using Dodt gradient contrast. The LC was identified in horizontal brainstem slices as a distinct, relatively translucent cluster of cells with exceptionally large somata, typically 20-30 μm in diameter. For current clamp recordings and voltage clamp recordings measuring K⁺ currents, patch pipettes (open pipette resistance 1.6-2.2 MO) were filled with an internal solution containing (in mM): 135 KMeSO₄, 5 KCl, 5 HEPES, 1.1 EGTA, 4 MgATP, and 0.3 Na₂GTP, and 10 Na₂Creatine Phosphate (pH 7.25, osmolarity 286). For the experiments summarized in FIG. 6, 20 μM Alexa 594 (Molecular Probes) was included in the internal solution.

Recordings were made with an Axoclamp 200B amplifier (Axon Instruments, Union City, Calif.). Data were filtered at 5 kHz and sampled at 10 kHz. Cells were held at −55 mV in voltage clamp mode, and no current was injected in current clamp mode. Cells were rejected if holding currents exceeded −200 pA. Series and input resistance were measured throughout the experiment, and recordings were discarded if series resistance exceeded 15 MO. Liquid junction potentials of −8 mV were not corrected except when calculating the reversal potential in FIG. 4. In these experiments we only accepted recordings in which series resistance was between 5-8 MΩ and compensation was not applied.

### Pharmacology

In all experiments, the following pharmacological agents were used in the extracellular solution at the following final concentrations (in μM): 10 CPP (Tocris), 10 NBQX (Tocris), 25 picrotoxin (Tocris). In some experiments, additional agents were added, as indicated in the text: 2 μM naloxone (Tocris), 100 μM carbenoxolone (Tocris), 3 μM thiorphan (Sigma), 20 μM bestatin (Sigma), 3.5 mM BaCl₂ (Sigma), 1 μM TTX (Tocris), 1 mM 4-AP (Tocris), 300 μM CdCl₂ (Sigma), and 50 μM ZD7288 (Tocris).

### UV Uncaging

For UV uncaging, we used a custom setup based on a BX51W1 microscope (Olympus). The output of a 100 KHz pulsed q-switched UV laser (Model 3501, DPSS, Santa Clara, Calif.) producing ˜800 mW of 354.7 nm light was launched into a multimode, 200 μm inner diameter optical fiber with a numerical aperture of 0.22 (OZ Optics, Ottawa, Ontario, Canada). The beam was shuttered at the laser head (OZ Optics, part number HPUC-2,A3HP-355-M-10BQ-1-SH) and collimated at the output of the fiber using either a factory (OZ Optics, part number HPUC0-2,A3HP-355-M-25BQ) or custom built collimator to produce a 10 mm diameter beam. Laser pulses were controlled by opening the shutter, waiting for mechanical vibrations in the fiber launch to dampen, and then q-switching the laser on and off. Light power levels were monitored with a PDA25K amplified photodiode (Thorlabs). Uncaging areas were measured by imaging laser-evoked fluorescence from a thin layer of an aqueous fluorescein solution that was sandwiched between two glass coverslips and placed in the sample chamber.

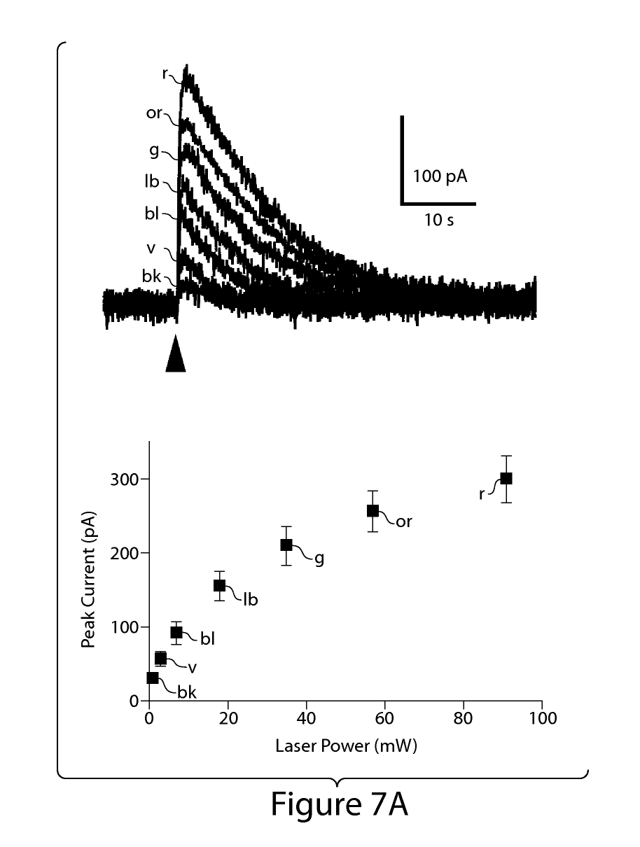

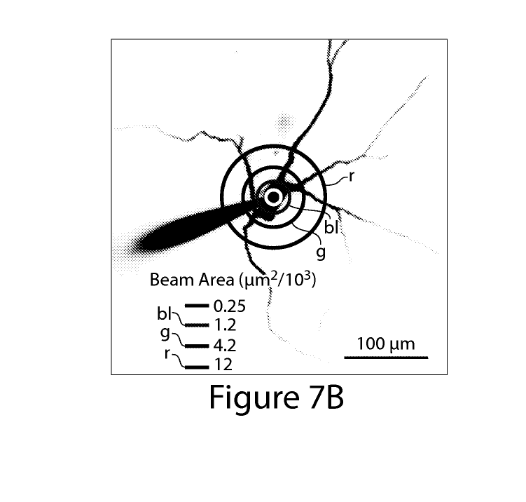

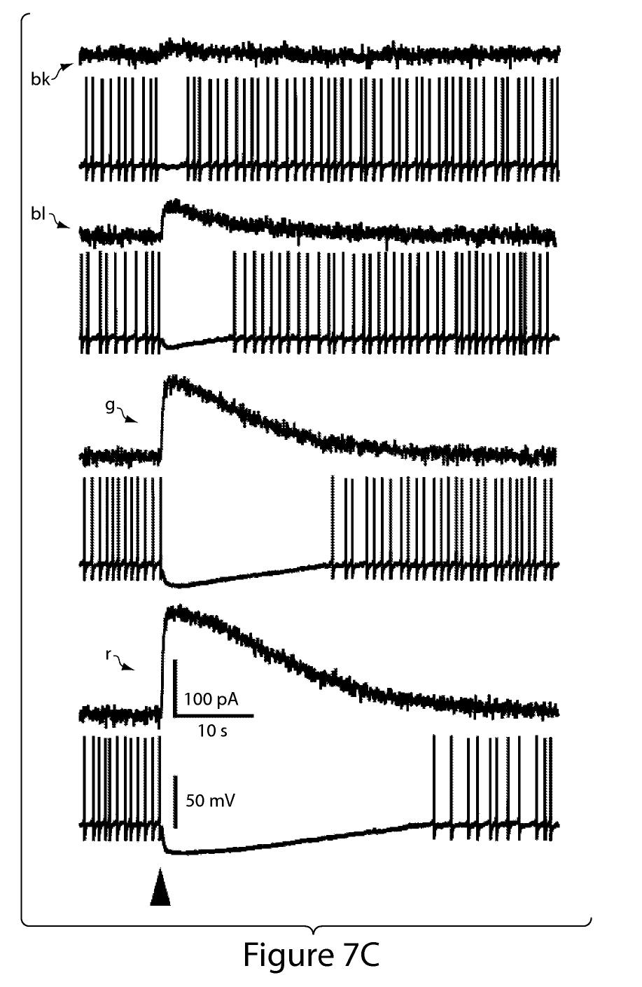

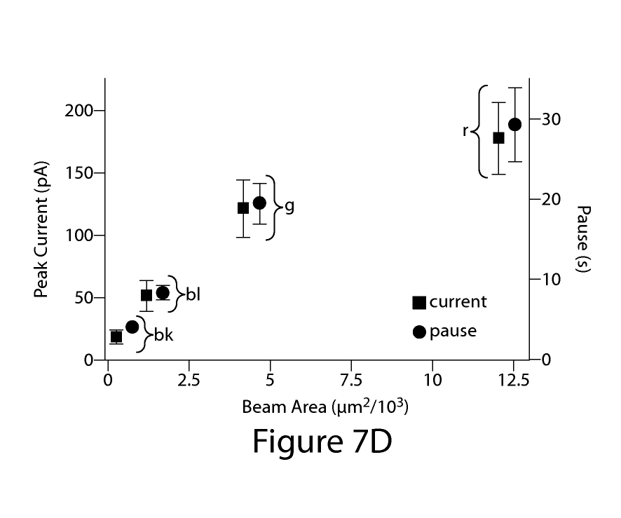

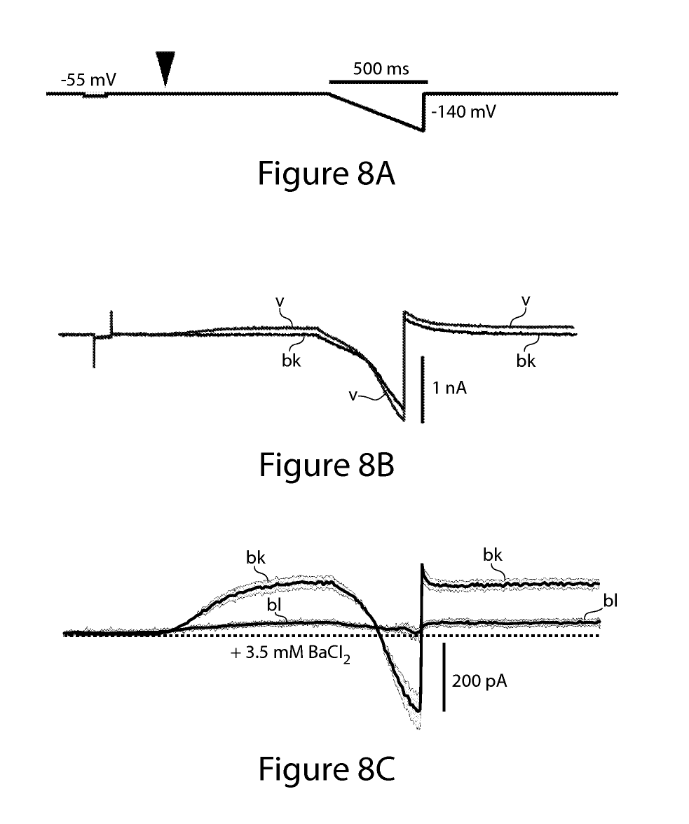

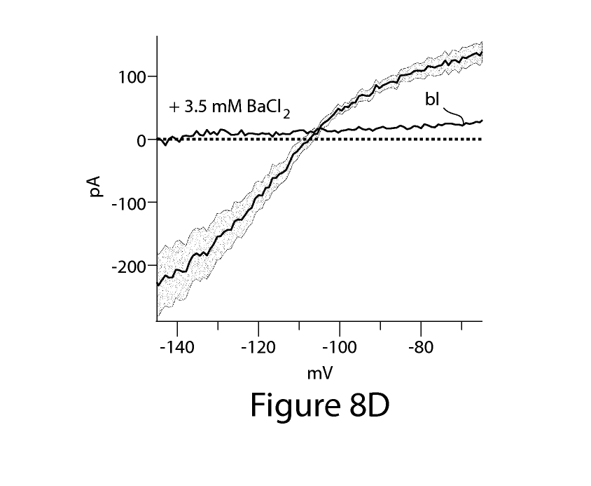

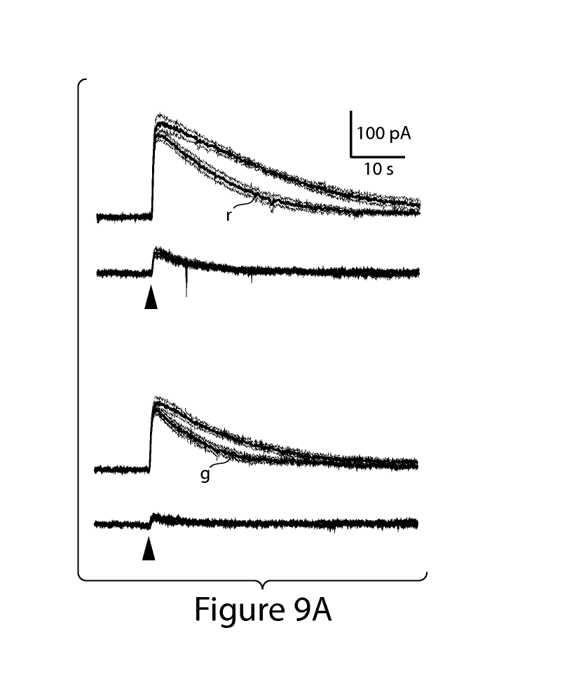

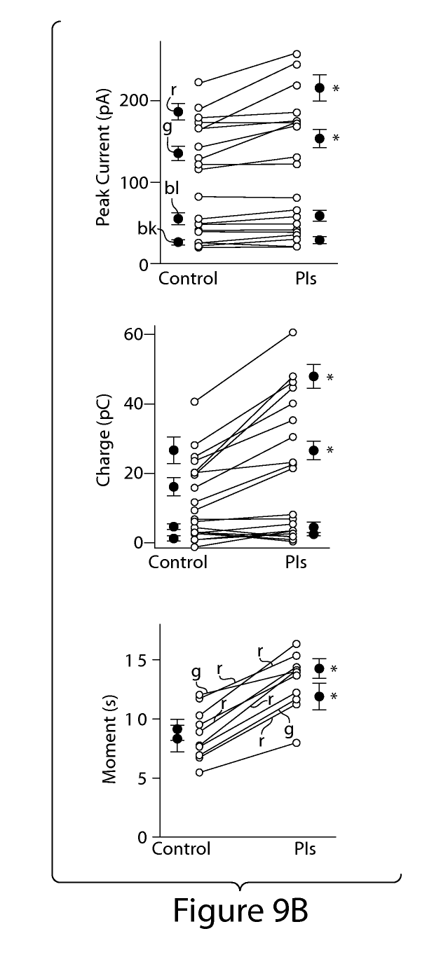

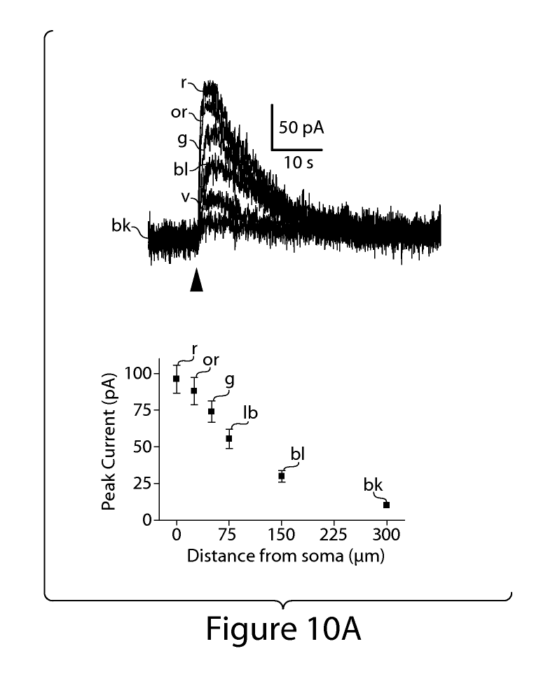

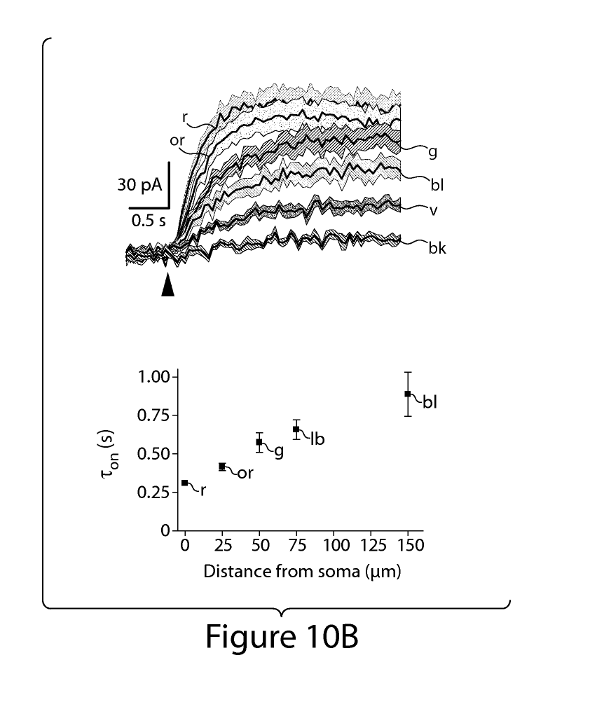

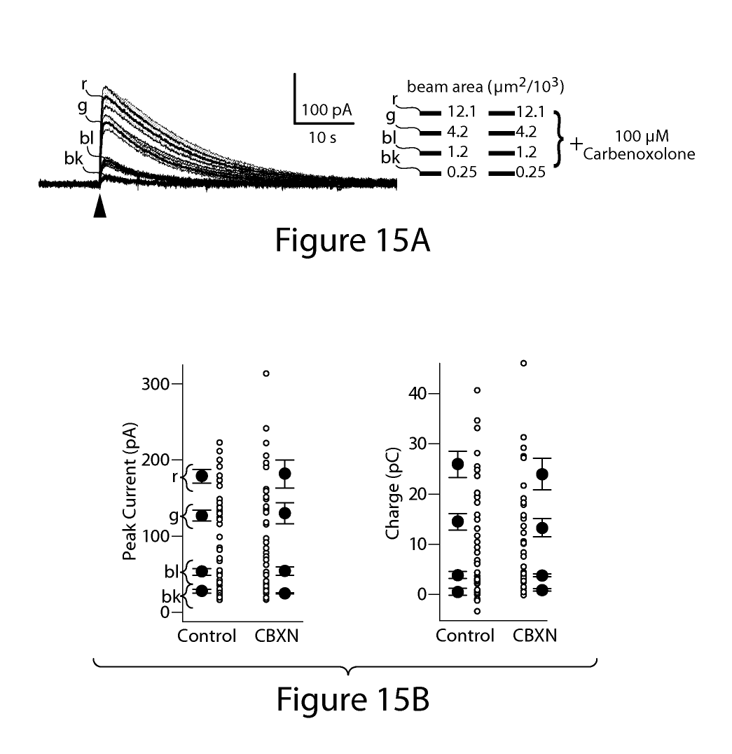

For the experiments in FIG. 6, FIG. 7B-D, FIG. 8 and FIG. 9, the 10 mm diameter beam was focused using a planoconvex lens onto the back focal plane of a 60× water-immersion, infinity-corrected objective with a numerical aperture of 0.90 (Olympus) to produce a collimated beam of ˜124 μm in diameter. Light intensity was attenuated to ˜25 mW, measured as a 10 mm diameter beam at the back aperture of the objective with the planoconvex lens removed from the light path. An iris placed in the light path in a conjugate image plane served as a field diaphragm. The iris was adjusted such that the diameter of the area in the tissue exposed to UV light was either ˜124 μm, ˜73 μm, ˜39 μm or ˜18 μm, corresponding to the beam areas of 12·10³ μm², 4.2·10³ μm², 1.2·10³ μm² or 250 μm², respectively, as indicated in the text. For the experiments in FIG. 7A, the beam was launched directly into the objective to produce a focused UV spot of ˜30 μm in diameter and power was modulated with neutral density filters to range from 1 mW to 91 mW, measured as a 10 mm diameter beam at the back aperture of the objective. In this optical configuration, photolysis at light intensities greater than 91 mW led to unstable recordings. For the experiments in FIG. 10, the output from a multimode, 25 μm inner diameter optical fiber with an numerical aperture of 0.13 (OZ Optics, Ottawa, Ontario, Canada) was collimated to a 10 mm diameter beam and launched directly into the objective to produce a focused UV spot of ˜2 μm in diameter at the sample. Power was modulated empirically to yield a ˜100 pA response at the soma. For the experiments in FIG. 15, the output from the μm fiber was collimated to a 2.5 mm diameter beam that was focused using a planoconvex lens onto the back focal plane of the objective. The field diaphragm was adjusted to produce a collimated beam of 10 μm in diameter at the sample.

### Two-Photon Imaging

Neurons in acute slices were loaded with 100 μm Alexa Fluor 594 via a patch pipette, and images were taken using a custom-built 2-photon microscope and a Chameleon Ti-Sapphire laser tuned to 840 nm.

### Data Analysis and Statistics

All data are expressed as the mean±SEM. In the figures, average traces are shown as the mean (line)±the SEM (shaded regions or bars). A two-tailed t test was used to determine significance of differences across conditions in FIG. 6 and FIG. 9. p<0.05 was considered significant.

### Further Neuropeptide Examples

The photoactivation studies are extending to additional caged peptides such as Dynorphin, Substance P and Cholecystokinin Activation/response studies for these neuropeptides will focus on the basal ganglia, which comprise a neural network involved in motor control and behavioral reinforcement. Although the basal ganglia are rich in Enkephalin, Dynorphin and Substance P, the roles of these peptides in circuit function are very poorly understood. The basal ganglia studies will combine neuropeptide uncaging with optogenetics and two-photon uncaging of glutamate and GABA to study the modulation of defined cells and synapses by the released neuropeptides. To facilitate these studies, caging groups with altered photolysis wavelength and two-photon sensitivity are also being explored. When applied to in vivo physiology studies, light pulses will trigger the rapid and spatially delimited release of neuropeptides in brain tissue, and neurons will be studied with electrophysiological and imaging approaches during exposure to the activated neuropeptides, enabling detection of the acute effects of peptides on cellular and synaptic physiology. Thus, the follow-on studies hypothesize strong direct responses of cells to small neuropeptides, and are intended to explore, confirm or elucidate the role of these neuropeptides as modulators of the electrophysiological behavior, chemical responses and other activity of neurons and synapses, and determine different aspects of neurotransmission in several subsets of synapses affected by each neuropeptide.

### First Stage, Caging

The approach described herein, based on the design and synthesis of photoactivable peptides, are believed to be new and never before employed for the study of neuropeptide signaling.

Once a photoactivatable neuropeptide has been created and validated, it can be quantitatively applied to brain tissue by using pulses of light to rapidly and selectively expose neurons to the biologically active neuropeptides. By coupling this method of delivery to traditional electrophysiological and imaging analysis of neuronal function one is then able to determine roles of neuropeptides in regulating neuron and synapse function.

Photoactivatable or “caged” biologically relevant molecules are known in some areas, and have been used previously (Ellis-Davies, G. C. R. (2007) Nature Methods 4, 619-628). In order to render a molecule photoactivable, a modification is made to the molecule that renders it active. This is typically done by adding a large side group to a portion of the molecule that is known to participate in the interaction with its binding partner or receptor. This side group must be linked to the parent molecule by a photosensitive bond that is rapidly cleaved on exposure to light. Lastly, the caging side group, once it is separated from the parent molecule, must be non toxic and biologically inert.

This general approach was previously applied to MNI-glutamate, a version of caged glutamate used in Applicant Sabatini's laboratory wherein caging was accomplished by the addition of a group to the terminal carboxylic acid (Carter A. G. et al. (2007) J Neurosci 27, 8967-8977; Bloodgood B. L. et al. (2007) Neuron 53, 249-60; Soler-Llavina G. J. et al. (2006) Nat Neurosci 9, 798-806; Bloodgood B. L. et al. (2005) Science 310, 866-9; Carter A. G. et al. (2004) Neuron 44, 483-493; Matsuzaki M. et al. (2001) Nat Neurosci 4, 1086-92). This side group, as for most photoactivatable molecules, is aromatic and absorbs light in the ultraviolet (UV) range, or by 2-photon mediated absorption, in the near-infrared red (NIR) range. Applicants have used 2-photon laser photoactivation (2PLP) of this molecule combined with 2-photon laser scanning microscopy (2PLSM) and electro-physiological recordings to examine the regulation of the electrical and biochemical consequences of synaptic transmission in several brain regions (Carter A. G. et al. (2004) Neuron 44, 483-493 and Alvarez V. A. et al. (2007) J Neurosci 27, 7365-76).

Caging of peptides and proteins with photo-removable protecting groups is usually done by covalently attaching a 2-nitrobenzyl moiety to a heteroatom (X) on the peptide such as N, O, or S. Upon absorption of UV light, the nitro group abstracts a benzylic proton to initiate a series of rearrangements that forms a 2-nitrobenzaldehyde and releases the caged heteroatom. Most caged peptides contain nitrobenzyl groups attached to amino acid side chains involved in receptor recognition and provide steric bulk that reduces hydrogen bonding and decreases peptide to receptor affinity. Peptides have also been protected at backbone amide nitrogens to provide excess steric bulk and prevent biologically active conformations. Backbone caging is less common but potentially more general because recognition sequences may not always contain chemically modifiable polar side chains. Both side chain and backbone caging may be used to prepare biologically inactive neuropeptides and their analogs for the practice of this invention.

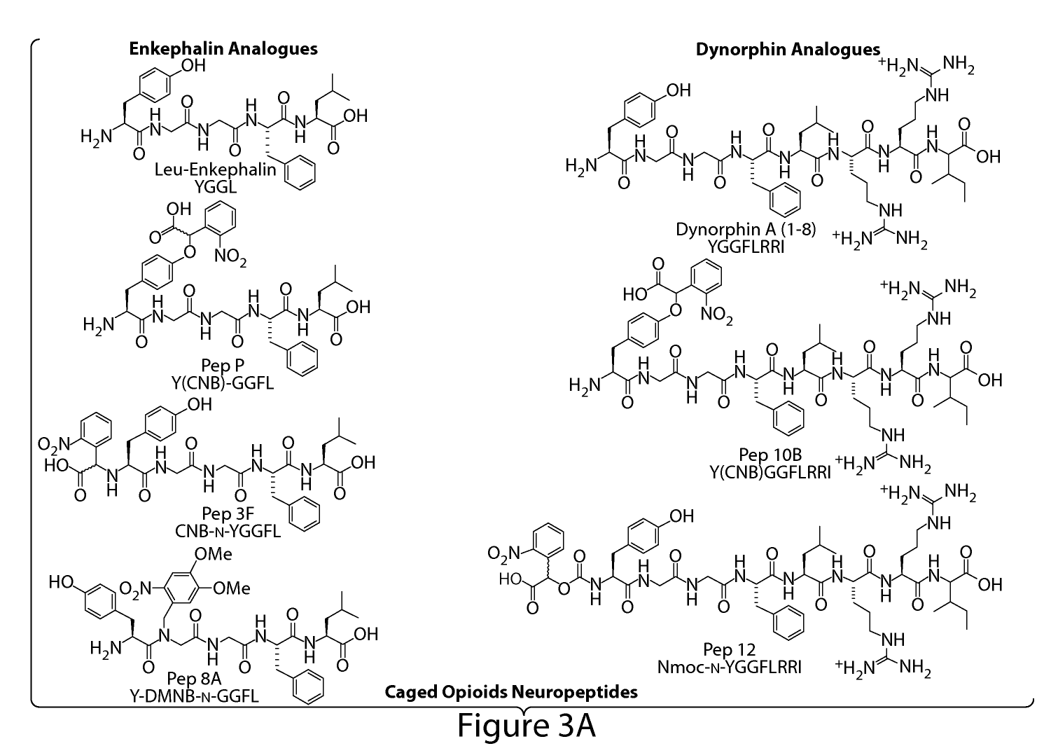

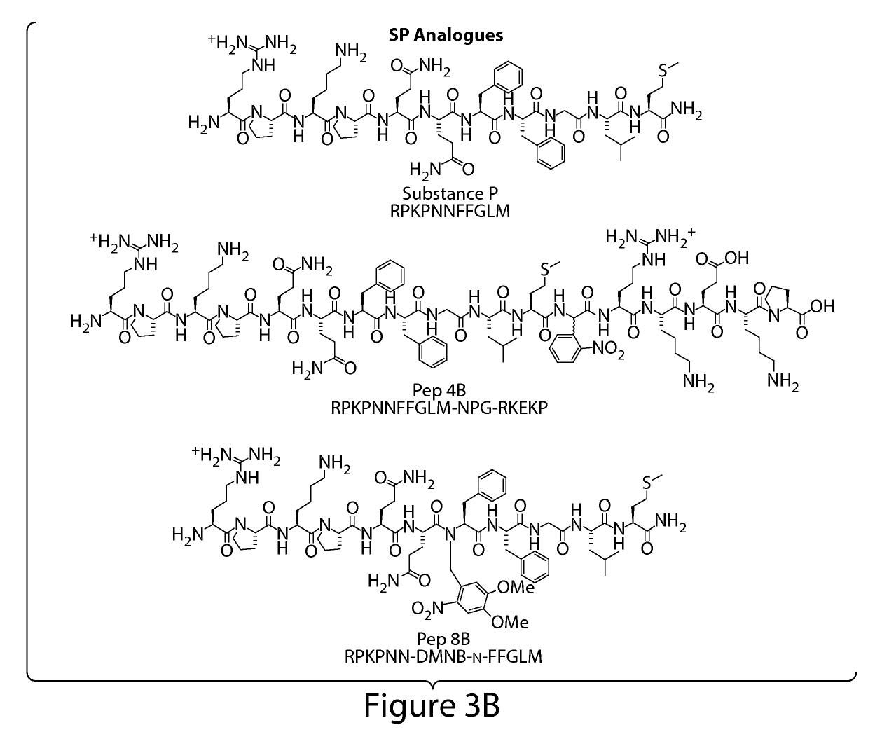

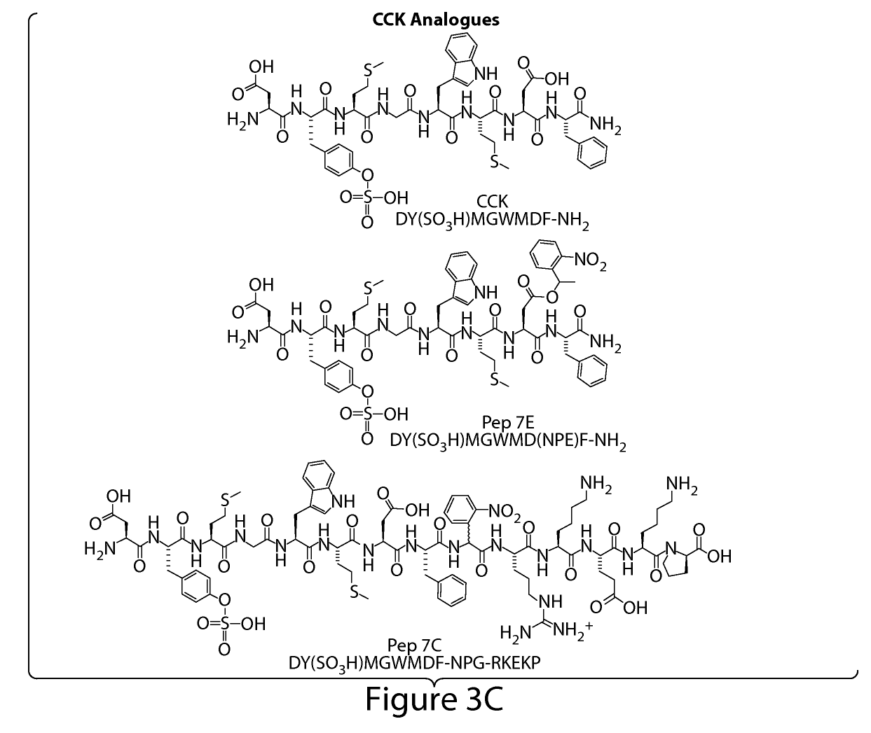

Of Substance P's eleven amino acids RPKPNNFFGLM (SEQ ID NO: 3), amino acids 6-11 NFFGLM (SEQ ID NO: 4) comprise the key recognition sequence (FIG. 3B) (Quartara L. et al. (1997) Neuropeptides (Edinburgh) 31, 537-563). To cage substance P or an analogue thereof, a dimethoxynitrobenzyl (DMNB) group may be appended to the side chain of glutamine 6 as the rest contain nonpolar side chains. The backbone amide nitrogen atoms of residues 6-11 may each be protected with DMNB groups, one at a time. Compared to the standard 2-nitrobenzyl caging group, DMNB releases twice as much photolysis product when irradiated with a standard 355 nm laser line. (Chang C. Y. et al. (1998) Journal of the American Chemical Society 120, 7661-7662). Applicants thus expect improved resolution and experimental power to be achievable with DMNB groups as compared to the nitrobenzyl cage employed on NMI-glutamate.

Furthermore, 2-photon uncaging of the DMNB chromophore can be performed with 700-750 nm wavelength light, as currently used in the lab for photoactivation of MNI glutamate, as in Aujard I. et al. (2006) Chemistry—A European Journal 12, 6865-6879. Caging the C-terminal amide is also a possibility. For the opioid peptides, it is known that the phenolic proton on the N-terminal tyrosine is necessary for receptor recognition. O-alkylation produces peptides with dramatically reduced affinity and no sign of antagonism. This position will be caged first (Dolle R. E. et al. (2007) Bioorganic & Medicinal Chemistry Letters 17, 2656-2660; Hruby V. J. et al. (1989) Med Res Rev 9, 343-401).

As described herein, DMNB can be attached at this crucial tyrosine. Synthesis of peptide analogues may be performed using solid-phase peptide synthesis (SPSS) using standard Fmoc chemistry and BOP coupling, which has proven compatible with DMNB chemistry (Nandy S. K. et al. (2007) Organic Letters 9, 2249-2252).

### Stage 2: Validation of Caged Neuropeptides Using Heterologous Systems

Once candidate photoactivatable neuropeptides have been synthesized, the biological inertness of the caged compound and its ability to release active peptide are tested, e.g., using a calcium mobilization assay in a heterologous cell system. Briefly, the receptors for most neuropeptides are G-protein coupled receptors (GPCRs) which have been cloned and are available from public libraries for a nominal fee. See, e.g., http://www.cdna.org/home.php?cat=8.

Each receptor is expressed in a cell line such as CHO cells along with a promiscuous alpha subunit of the Gprotein (e.g. Ga16) that couples activation of the GPCR to release of calcium from intracellular stores. Using this system, exposure of the cell to the peptide ligand triggers larges increases in intracellular calcium concentration that can be detected easily using fluorescence based calcium indicators. This assay is used quantitatively to determine the receptor affinity of the caged parent compound and the efficiency of release of the active peptide. In addition, the laser pulse parameters necessary for photoactivation of caged neuropeptides are determined.

### Stage 3: Rapid Effects on, Neuronal Physiological of Neuropeptide Receptor Activation

Following validation in the heterologous cell system, the effects of photoactivation of caged neuropeptides on neuronal physiology are examined, for example using acute brain slices of rat striatum and the hippocampus as our experimental preparations. Applicants have extensive experience performing electrophysiological and imaging studies in these areas and have used 2-photon mediated photolysis of caged glutamate to study synaptic transmission in both brain areas See above citations and also Ngo-Anh T. J. et al. (2005) Nat Neurosci 8, 642-9; Shankar G. M. et al. (2007) J Neurosci 27, 2866-75; and Tavazoie S. F. et al. (2005) Nat Neurosci 8, 1727-34). Whole-cell recordings from neurons in the brain slice will be used to monitor electrical signaling in the cell. In addition, by loading neurons with fluorophores and imaging the neurons under 2PLSM Applicants are able to analyze the morphology of the cell and monitor changes in intracelluluar calcium levels. (See the referenced Appendix A)

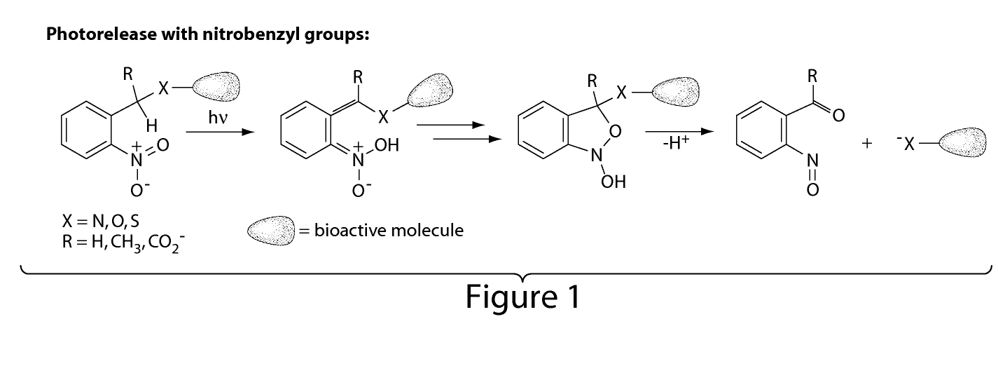

In the experimental protocol, caged neuropeptides are applied to the bathing medium and pulses of UV or NIR light are used to trigger 1-photon or 2-photon, respectively, mediated photolysis of the compound. Parameters to be monitored include the effects of peptide release on resting membrane properties of the cell, action potential initiation and propagation, synaptic transmission, intracellular calcium signaling, and morphological plasticity. This battery of assays is well established in the laboratory and covers many of the dynamical facets of neuronal function. By caging small neuropeptides and arranging defined and controlled activation by photolysis, Applicants thus provide tools of great utility and precision for studying the mechanisms of action of neuropeptides, and provide for the first time a body of observation on peptide-based neurotransmission and neuromodulation. By integrating these observations with known aspects of synaptic transmission and its regulation in a variety of brain areas including hippocampus, cerebellum, and striatum, and applying Applicants' experience with optical and electrophysiological studies of synapse structure and function, further practical applications to medical neurology and pharmacology are anticipated, including novel materials and treatments for pain, behavioral and psychological conditions. The studies build upon earlier investigations that combined 2-photon laser scanning microscopy (2PLSM) and 2-photon laser photoactivation (2PLP) of light sensitive molecules (FIG. 1) to uncover mechanisms acting within about one micron of active synapses to locally regulate the biochemical and electrical consequences of synapse activation. Those investigations led to a new understanding of mechanisms that allow independent regulation of individual synapses in neurons that lack dendritic spines. By directly monitoring and manipulating biological signals within the individual spine heads they also revealed signaling cascades that act entirely within these small compartments to regulate synaptic signaling. Those investigations also included the first analysis of calcium signaling in individual spines of striatal MSNs and found that electrophysiological state-transitions regulate synaptic signals and fundamentally alter the integrative properties of the neuron. The use of photoactivated caged neuropeptides as described above presents new tools and methods of analysis for the study of human neurological diseases, which may, for example be applied to the perturbed synapse structure and function in a mouse model of Tuberous Sclerosis Complex elucidated in Tavazoie, S. F. et al. (2005) Nat Neurosci 8, 1727-34; or to the cellular and synaptic perturbations triggered by pathogenic forms of amyloid-β reported in Shankar, G. M. et al. (2007) J Neurosci 27, 2866-75 as well as the electrophysiological responses reported in Carter, A. G. et al. (2007) J Neurosci 27, 8967-77 and Carter, A. G. et al. (2004) Neuron 44, 483-93. By adding the caged neuropeptides response analysis to the optical investigation of acetylcholine and dopamine modulation of synapses within the striatum and trafficking of proteins into and out of individual synapses in real-time Applicants can for the first time detect and quantify previously inaccessible secondary and complex signaling mechanisms and pathways in synaptic structures. These tools may also be combined with electrophysiological and imaging approaches with genetic manipulations to examine the roles of disease-related proteins in regulating the structure and function of neurons and synapses.

Beyond proof-of-principle experiments involving the design, caging and synthesis of a caged neuropeptide, optimal practice of the invention is achieved by optimization of the caging groups and the uncaging protocol. Work is preferably performed in heterologous expression systems such as neuropeptide receptor transfected CHO cells. Extensive data gathering is then done testing the effects of rapid release of caged neuropeptide in neurons. Design and validation of second generation neuropeptides, as well as further optimization of caging groups and protocols may be performed simultaneously to develop a suitable number of neuropeptides and analogues for carrying forward the investigation of known processes. This leads to testing and use of caged neuropeptides in brain slices, and mechanistic analysis of the effects of rapid neuropeptide release on synaptic transmission and neuronal function in the hippocampus and striatum.

Caged neuropeptides are further described in the cited Appendices A-E of above-referenced provisional application 61/415,730. Appendix A is a 22-page power point providing a schematic overview of candidate neuropeptides and initial selection, and a series of protocols for their use in elucidating corresponding neuropeptide function and measuring synaptic responses and the activities extending across regions of the brain. Appendix B is a related presentation Nov. 18, 2010, of experimental results confirming the effectiveness of neuropeptide caging and dynamics of photorelease, and effects on release in acute brain slices; also showing the absence of antagonism of the caged analogue; and demonstrating certain factors affecting dosing, spatial control and cellular response. Appendix C is a brief description including images and synaptic current traces obtained by activating caged MNI glutamate at positions on a spiny dendrite, establishing the use of such caged neurotransmitter molecules for quantitative measurement of a response such as calcium influx to application of the MNI glutamate at various sites of a synapse. Appendix D describes an enhanced SEAP assay employed for detecting and quantifying the response to localized application of a neuropeptide according to the invention; and Appendix E illustrates caging approaches for forming caged neuropeptides as described herein.

### Further Considerations, Measurements of Activity

### Design of Photoactivatable Opioid Neuropeptides

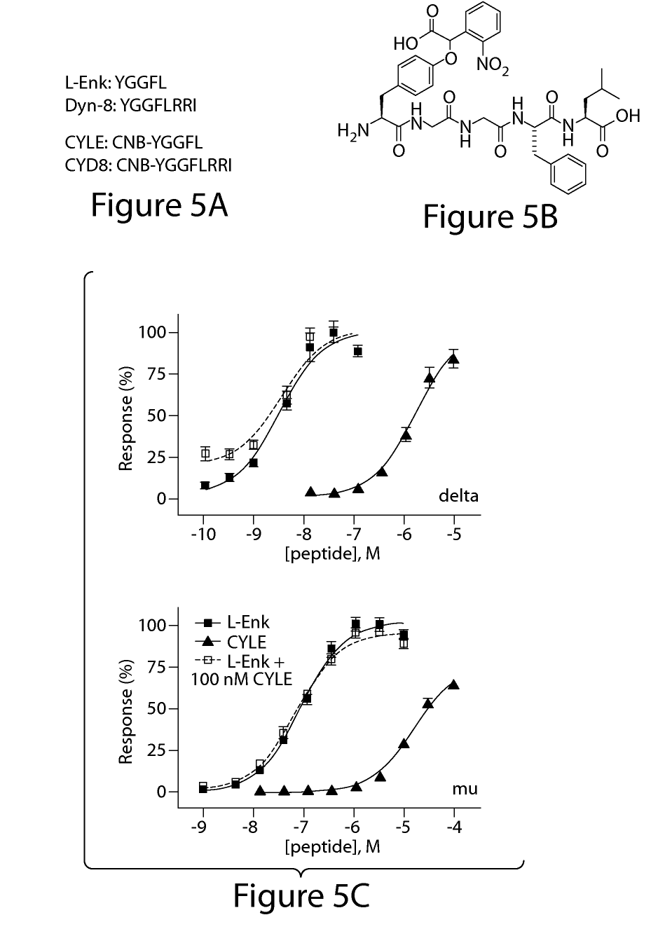

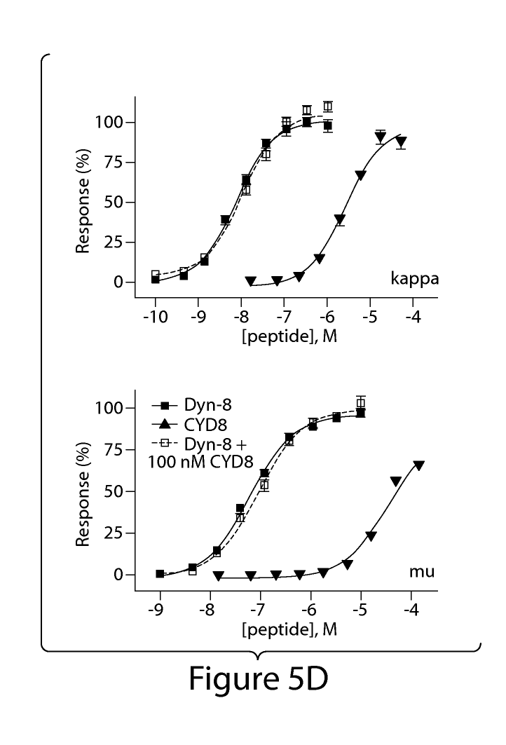

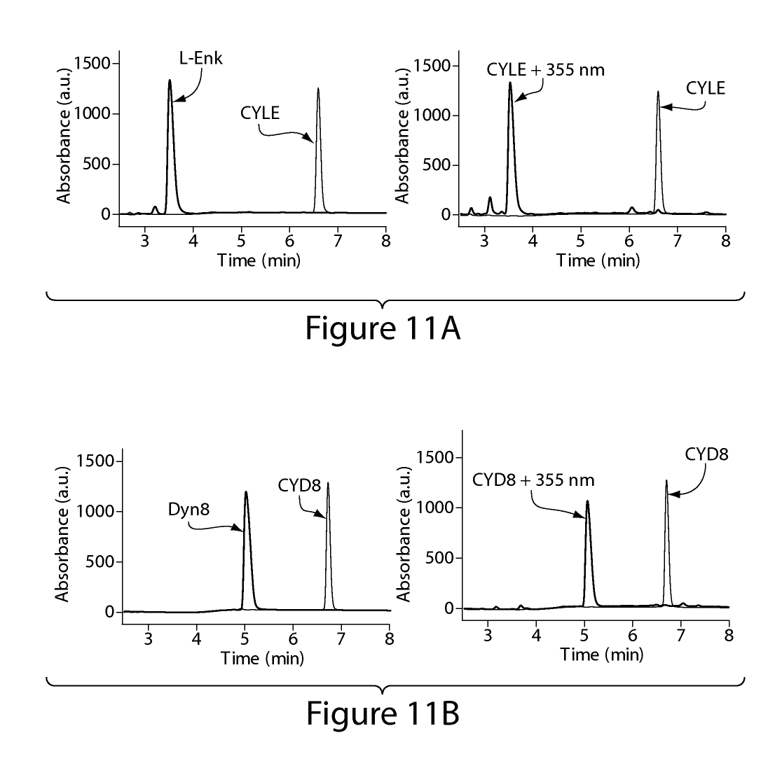

Enkephalins and dynorphins are the most prominent opioid peptides in the brain (Khachaturian, H. et al. (1985) Trends Neurosci 8, 111-119). We chose to work with L-Enk and Dyn-8 (FIG. 5A) because they are the smallest and most chemically stable endogenous opioids from these peptide families. L-Enk activates delta and mu opioid receptors with nanomolar affinity, but is inactive at kappa receptors (Toll, L. et al. (1998) NIDA Res Monogr 178, 440-466). The three additional C-terminal amino acids found in Dyn-8 confer nanomolar potency at kappa receptors in addition to mu and delta receptors (Toll, L. et al. (1998) NIDA Res Monogr 178, 440-466). To render these peptides inactive until exposed to light, we produced analogues modified at the N-terminal tyrosine side chain with the carboxynitrobenzyl (CNB) chromophore, which photo-releases tyrosine with high quantum yield (˜0.3) (Sreekumar, R. et al. (1998) Methods Enzymol 291, 78-94) on the microsecond time scale (Tatsu, Y. et al. (1996) Biochemical and Biophysical Research Communications 227, 688-693) (See Supporting Information for information on peptide production and handling). Extensive studies into the structure-activity relationships of enkephalins (Morley, J. S. (1980) Annual Review of Pharmacology and Toxicology 20, 81-110) and dynorphins (Chavkin, C. et al. (1981) Proc Natl. Acad Sci USA 78, 6543-6547) have revealed an essential role for their common N-terminal tyrosine (Y) in receptor activation. In particular, alkylation (Beddell, C. R. et al. (1977) Proc R Soc Lond B Biol Sci 198, 249-265) or removal (Terenius, L. et al. (1976) Biochem Biophys Res Commun 71, 175-179) of the phenolic OH group reduces the potency of enkephalin analogues, suggesting that modification of the tyrosine side chain of L-Enk and Dyn-8 may be a viable caging strategy. Based on these considerations, we designed CNB-Y-Leucine-Enkephalin (CYLE) and CNB-Y-Dyn-8 (CYD8) (FIG. 1A) to release L-Enk and Dyn-8, respectively, in response to illumination with ultraviolet (UV) light. The chemical structure of CYLE is shown in FIG. 5B. Reverse-phase high pressure liquid chromatography experiments confirmed that both peptides cleanly photorelease their parent peptides in pH 7.4 phosphate buffered saline in response to 355 nm laser illumination as shown in FIG. 11, and that they are stable in the dark at room temperature for >48 hrs (data not shown).

### Activity of CYLE and CYD8 at Opioid Receptors

To determine if CYLE and CYD8 are inactive at opioid receptors prior to photolysis, we compared their activity on opioid receptors relative to that of L-Enk and Dyn-8, respectively, using an in vitro functional cellular assay. To detect opioid receptor activation, we utilized HEK293 cells that stably express a Gαₛ-Gαᵢ chimera (Liberles, S. D. et al. A High Throughput Assay for GPCR Ligands Using G(alpha)s Chimeras. Submitted). This chimeric protein allows GPCRs that normally do not signal through Gαₛ to stimulate adenylate cyclase and control the transcription of a cAMP-dependent reporter construct. Cells were co-transfected with the opioid receptor of interest and the reporter construct such that receptor activation leads to production of secreted alkaline phosphatase (SEAP). The amount of SEAP that accumulates in the media is assayed on a fluorescence plate reader with the fluorogenic substrate 4-methylumbelliferone phosphate (Liberles, S. D. et al. (2006) Nature 442, 645-650).

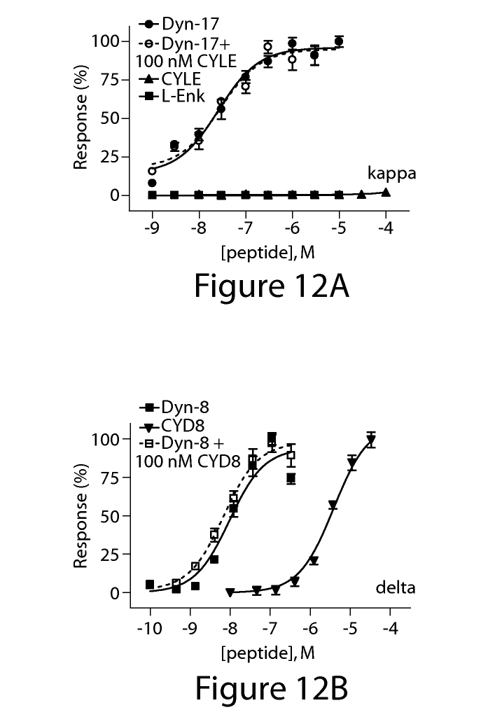

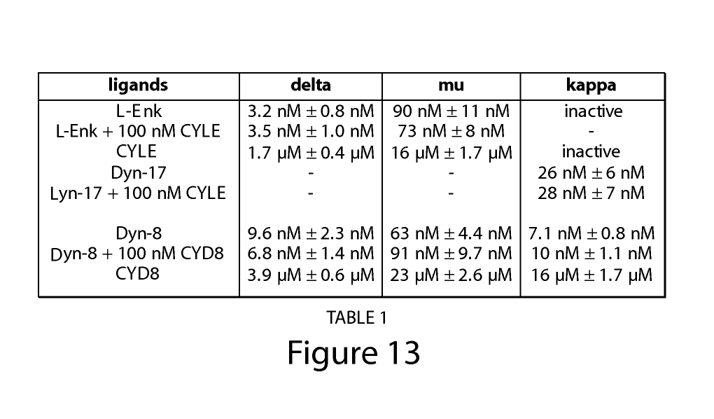

Using this assay, we obtained dose-response curves for CYLE and CYD8 at mu, delta and kappa opioid receptors and compared those to the actions of the parent peptides (FIG. 5C, D and FIG. 12). FIG. 5C shows dose-response curves for L-Enk, CYLE and L-Enk in 100 nM CYLE at delta (left) and mu (right) opioid receptors. The solid lines depict the best fit sigmoidal functions used to derive the EC₅₀ values reported in the text. The dashed lines are fits to the data obtaining by adding caged compound to the parent peptide dilution series and demonstrate the lack of antagonist by the caged compound. Data were normalized to the maximal responses produced by the endogenous peptide agonists. Similarly, FIG. 5D shows the dose-response curves for Dyn-8, CYD8, and Dyn-8 in 100 nM CYD8 at kappa (left) and mu (right) opioid receptors. The potency of CYLE was reduced 100-500 fold with respect to L-Enk at both delta (L-Enk EC₅₀: 3.2 nM±0.8 nM, CYLE EC₅₀: 1.7 μM±0.4 μM) and mu receptors (L-Enk EC₅₀: 90 nM±11 nM, CYLE EC₅₀: 16 μM±1.7 μM, FIG. 5D). Similar to L-Enk, CYLE did not activate kappa receptors (FIG. 12) and the presence 100 nM CYLE did not reduce the affinity of L-Enk at mu and delta receptors (FIG. 5C) or that of Dyn-17 at kappa receptors (FIG. 12), indicating that CYLE does not act as an antagonist. CYD8 exhibited similar reductions in potency in comparison to Dyn-8 at kappa (Dyn-8 EC₅₀: 7.1 nM±0.8 nM, CYD8 EC₅₀: 16 μM±1.7 μM), mu (Dyn-8 EC₅₀: 63 nM±4.4 nM, CYD8 EC₅₀: 23 μM±2.6 μM, FIG. 5D) and delta receptors (Dyn-8 EC₅₀: 9.6 nM±2.3 nM, CYD8 EC₅₀: 3.9 μM±0.6 μM, FIG. 12), with no indications of antagonism. Summary dose-response data are tabulated in Table 1 of FIG. 13. These data reveal that CYLE and CYD8 possess no significant agonist or antagonist activity at concentrations that should strongly activate receptors following photolysis.

### Photorelease of Opioids in Brain Slices

In order to characterize the ability of these molecules to activate neuropeptide receptors in brain slices with spatiotemporal precision, we took advantage of the well-described opioid receptor-mediated activation of K⁺ channels in neurons of the locus coeruleus (LC). The LC is heavily innervated by enkephalinergic afferents (Curtis, A. L. et al. (2001) J Neurosci 21, RC152; Drolet, G. et al. (1992) J Neurosci 12, 3162-3174) and LC neurons express a high density of mu opioid receptors in their somata and dendrites (Van Bockstaele, E. J. et al. (1996a) J Neurosci 16, 5037-5048; Van Bockstaele, E. J. et al. (1996b) J Comp Neurol 376, 65-74), activation of which pauses spontaneous firing (Pepper, C. M. et al. (1980) Science 209, 394-395) by producing large outward currents (Travagli, R. A. et al. (1995) Journal of Neurophysiology 74, 519-528; Travagli, R. A. et al. (1996) J Neurophysiol 75, 2029-2035; Williams, J. T. et al. (1982) Nature 299, 74-77), at least in part through G-protein coupled inward rectifier K⁺ (GIRK) channels.

We obtained whole-cell recordings from neurons in acute horizontal slices of rat LC (FIG. 6A) and characterized the ability of CYLE to photoactivate mu receptors by releasing L-Enk. In current clamp recordings, these cells spontaneously fired action potentials at 2-8 Hz, while local perfusion of 10 μM L-Enk caused a strong hyperpolarization and transient pause in spiking (FIG. 6B), consistent with previous studies (Williams, J. T. et al. (1982) Nature 299, 74-77).

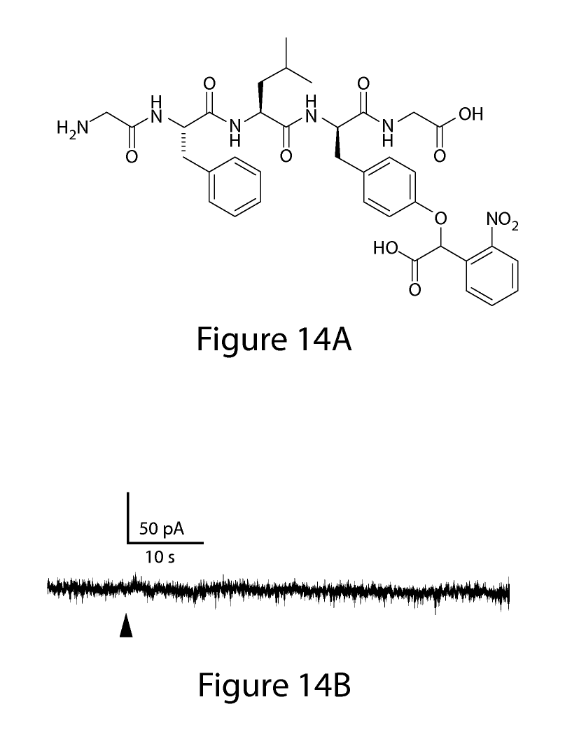

In voltage-clamp recordings at a holding potential of −55 mV, application of 10 μM L-Enk via a perfusion pipette placed near the cell of interest evoked average (n=6) outward currents of 199±23 pA in amplitude as shown in FIG. 6C. In contrast, when applied to the same cells 10 μM CYLE evoked an average outward current of 5.6±4 pA, measured 3.5-4 min after addition to the circulating ACSF (FIG. 6C). A subsequent uncaging stimulus consisting of a 5 ms flash from a 124 μm diameter beam of 355 nm light induced a large outward current that was blocked by the addition of 2 μM naloxone (NaI), a broad-spectrum opioid receptor antagonist as shown in FIG. 6D. Additionally, photolysis of an isomer of CYLE in which the amino acid sequence was scrambled to render it inactive at opioid receptors did not produce currents. FIG. 14 shows the peptide and flat signal trace.

To evaluate the extent and kinetics of photoactivation, we compared responses (n=6) evoked by local application of 10 μM L-Enk and a subsequent UV light flash in the presence of 10 μM CYLE (FIG. 6E). This analysis revealed that photorelease of L-Enk produces currents similar in amplitude to those evoked by the same concentration of locally applied L-Enk with peak current=207±19 pA versus 179±9 pA for local perfusion and photolysis, respectively (FIG. 6F). Nevertheless, consistent with rapid delivery of L-Enk directly to the recorded cell, the onset kinetics of the light-evoked response were nearly two orders of magnitude faster than for local perfusion (τₒₙ=349±26 ms versus 11.64±2.22 s for photolysis and local perfusion, respectively) (FIG. 6F), such that the peak current was reached within 1-2 s after the light flash. In contrast, the kinetics of deactivation for the uncaging response were only ˜2-fold faster (τ_{off}=24±2 s versus 14±1 s for local perfusion and photolysis, respectively) (FIG. 6F). Additionally, the responses to 15 uncaging stimuli delivered to the same cell once every 3 minutes were stable as shown in FIG. 6G. Thus CYLE enables rapid and robust delivery of enkephalin in brain slices.

One advantage of caged compounds is the ability to photorelease molecules in a graded or analog fashion by varying the amount of photolysis light. This can be readily achieved by manipulating the light intensity or the area of illumination. Various uncaging and response measurement events were examined, as shown in FIGS. 7-10. In the original rendering of FIG. 7 et seq. a spectrum of colors were used to represent different conditions, such as increasing parameter values, progressing in spectral order from black or violet (smallest value) to red (largest value) of a progression. Further, in related panels of the FIGURES, stimulus and response measurements which correspond to each other appear in the same color. In order to display this information in the black- and white FIGURES submitted herewith, the colors are indicated by letter-codes: black (bk), violet (v), blue (bl), light blue (lb), green (g), yellow (y), orange (or) and red (r) for the various illustrated parameters of laser power or ON time, beam area, distance from stoma, peak current and the like. Where light and dark hues of a single color are used, the corresponding feature in the FIGURE appears as dark or grey with a single color indication, as in FIG. 15A.

To explore the result of different light intensities, 5 ms flashes of a focused, 30 μm diameter spot of UV light were applied to the soma during voltage clamp recordings and varied the light intensity. Under these conditions, the light-evoked currents in individual cells increased in amplitude with light power (FIG. 7A) such that the average (n=8) peak currents ranged from 31±3 pA at 1 mW (black) to 300±32 pA at 91 mW (red). The right panel shows average peak amplitudes of currents evoked at different laser powers (n=6 cells). FIG. 7B shows a reverse contrast two-photon laser scanning microscopy image of a LC neuron filled with Alexa Fluor-594. The colored rings and corresponding legend indicate the various diameters of a collimated photolysis beam. FIG. 7C shows current (top) and voltage (bottom) responses of a LC neuron evoked by uncaging over the areas depicted in panel B; and FIG. 7D shows average peak current amplitude (left axis, squares) and action potential pause duration (right axis, circles) as a function of uncaging spot diameter. Circles are offset slightly to the right for clarity.

Another approach to analog delivery is to vary the area of illumination. This was achieved by shaping a collimated beam of fixed power density with a field diaphragm placed in a conjugate image plane of the laser path. The area of the field of illumination was varied from 250 μm², which is smaller than a typical LC cell body, to 12·10³ μm², which covers the soma and a large region of the proximal dendrites (FIG. 7B). Responses were measured in both voltage- and current-clamp recording configurations (FIG. 7C). In voltage-clamp, varying the area of illumination produced light-evoked outward currents of various magnitude (peak current=178±29 pA versus 18±6 pA for 12·10³ μM² and 250 μm² fields, respectively) and duration. In current clamp, the same uncaging stimuli produced pauses in spontaneous firing of graded duration (t=29±5 s versus 4±0.3 s for 12·10³ μm² and 250 μm² fields, respectively) and hyperpolarizations of graded amplitude. In individual cells and across cells, the responses to each photolysis condition in both recording configurations were tightly correlated (FIG. 7D). Furthermore, the onset kinetics of the light-evoked currents did not vary across the different uncaging stimuli (τₒₙ=349±26 ms versus 400±66 ms versus 417±112 ms for 12·10³ μm², 4.2·10³ μm² and 1.2·10³ μm² fields, respectively; one-way ANOVA p=0.81; kinetics could not be reliably derived for responses to the 250 μm² uncaging stimulus). These results indicate that photolysis delivers L-Enk directly to the site of action over a range of areas.

### The Ionic Basis of Enkephalin-Evoked Outward Currents

The ability to tightly regulate the area over which L-Enk is applied provides an opportunity to study the ionic conductances that underlie the mu opioid response in LC with unprecedented accuracy. Although it has been clearly demonstrated that mu opioid receptor activation opens GIRK channels in LC neurons (Torrecilla et al., 2002), reversal potentials determined for the evoked currents in brain slices are frequently much more negative (−140 mV to −120 mV) than predicted for a pure K⁺ conductance according to the Nernst equation (˜−105 mV typically). This observation might be accounted for by the inability to voltage clamp currents generated in the large (Shipley, M. T. et al. (1996) J Comp Neurol 365, 56-68), gap-junction-coupled dendrites (Ishimatsu, M. et al. (1996) Journal of Neuroscience 16, 5196-5204; Travagli, R. A. et al. (1995) Journal of Neurophysiology 74, 519-528) of LC neurons. Several studies suggest that inhibition of a standing, voltage-insensitive Na⁺ current may contribute 50% of the observed outward current response to enkephalin (Alreja, M. et al. (1993) Journal of Neuroscience 13, 3525-3532; Alreja, M et al. (1994) Brain Research 639, 320-324). Thus, the complete ionic nature of the enkephalin-evoked outward currents has been a subject of debate (Alreja, M. et al. (1993) Journal of Neuroscience 13, 3525-3532; Alreja, M et al. (1994) Brain Research 639, 320-324; Osborne, P. B. et al. (1996) Journal of Neurophysiology 76, 1559-1565; Torrecilla, M. et al. (2002) Journal of Neuroscience 22, 4328-4334; Travagli, R. A. et al. (1995) Journal of Neurophysiology 74, 519-528).

To address this issue, we measured the reversal potential of the L-Enk evoked outward current while restricting the uncaging area to the soma and proximal dendrites where voltage clamp is expected to be optimal (Williams, S. R. et al. (2008) Nat Neurosci 11, 790-798). Importantly, the responses to the uncaging stimuli shown in FIG. 7B were not significantly attenuated by the gap junction inhibitor carbenoxolone (FIG. 15), suggesting that gap junctions do not contribute to the L-Enk mediated currents evoked by uncaging CYLE around the soma. In FIG. 15 the data are color coded to indicate the uncaging stimuli as in FIG. 7. Panel A shows the average (n=10 cells) responses to the uncaging stimuli in the absence (standard colors) and presence (light colors) of the 100 uM carbenoxolone (CBXN). Panel B shows (left) peak amplitude; and (right) total charge transfer of the responses evoked by the uncaging stimuli for each cell (open circles). The average population data are also shown (closed circles).

To measure reversal potentials in the voltage range of a K⁺ conductance, we held cells at −55 mV and applied negative voltage ramps to −140 mV over 500 ms during the peak of the outward current (FIG. 8A). A response to the ramp alone (black) is presented with a response to the ramp after an uncaging stimulus (violet) in FIG. 8B corresponding to the 4.2·10³ μm² beam (the green ring in FIG. 6B). The average (n=12) L-Enk-evoked current (FIG. 8C, black trace) was isolated by subtracting the baseline from the light-evoked response. After correction for the junction potential this current reversed at −107.2±1.8 mV, closely matching the calculated K⁺ reversal potential of −106 mV ([K⁺]ₒᵤₜ=2.5 mM, ([K⁺]ᵢₙ=145 mM). In the presence of 3.5 mM BaCl₂, a GIRK channel blocker, the outward current (blue) was largely attenuated and only a small response remained (peak current=142±17 pA versus 29±4 for control and 3.5 mM BaCl₂, respectively). The average (n=13) L-Enk-evoked, Ba²⁺-insensitive current (FIG. 8C), appeared to reverse near −140 mV. We were unable to block or occlude this residual outward current with the voltage-sensitive Na⁺ channel blocker TTX, the voltage-gated K⁺ channel blocker 4-AP, the voltage gated Ca²⁺ channel blocker Cd²⁺, or the HCN channel blocker ZD7288 and it persisted in K free, Cs⁺ based internal (data not shown). Thus, at least 80% of the outward current evoked by L-Enk at the soma and proximal dendrites is carried by a Ba²⁺-sensitive K⁺ current.

### Proteolytic Contribution to Enkephalin Clearance in LC

Although neuropeptides are generally thought to be degraded by extracellular proteases, the extent to which this clearance mechanism limits the duration of peptide signaling after release is unknown. To determine the contribution of peptide degradation via proteolysis to the termination of enkephalin signaling, we repeated the voltage clamp experiment shown in FIG. 7B and compared the currents evoked by light before and after the addition of the peptidase inhibitors bestatin and thiorphan (FIG. 9A). This inhibitor combination blocks the degradation of enkephalin in brain tissue by >95% and increases the EC₅₀ of bath applied methionine-enkephalin by 7-fold in LC (Williams et al., 1987). FIGS. 9A-B show the effect of peptidase inhibitors on light-evoked currents. FIG. 9A shows responses to the uncaging stimuli before (colors) and after (gray) addition of the peptidase inhibitors, where the control condition traces are color-coded black/red/blue/green to indicate the uncaging beam area as in FIGS. 7B-D. FIG. 9B shows peak amplitude (left), total charge transfer (middle), temporal moment (right) of the responses evoked by the uncaging stimuli for each cell in basal control conditions and after addition of peptidase inhibitors (PIs). The average population data are also shown (closed circles). * denotes a significant difference from control. The small currents generated in response to the two smallest uncaging stimuli did not allow reliable calculation of moments.

When the uncaging beam was restricted to the soma (250 um² and 1.2 um²/10³ beam areas), no significant difference in the peak current, charge transfer or moment (the time at which half of the total charge transfer occurs) of the uncaging response was observed (FIG. 9B). This indicates that when opioids are released with spatial heterogeneity, the local response is determined by the local time course of peptide release and diffusion, without contribution of peptidase-mediated degradation. However, with larger uncaging areas, peptidase inhibition significantly enhanced all of these parameters. The total charge transfer at the largest area examined was particularly sensitive to this manipulation, as it was enhanced 1.8 fold, while the moment was enhanced 1.6 fold. The moment was used to quantify the deactivation time course because the decay kinetics in peptidase inhibitors were not well fit by a monoexponential, as expected from the complex kinetics of buffered-diffusion reactions. These results indicate that peptidases limit the spread of enkephalin signaling when released in large volumes but that diffusion plays a larger role in limiting the spread from spatially confined release sites.

### Spatial Limit of Enkephalin Signaling in LC

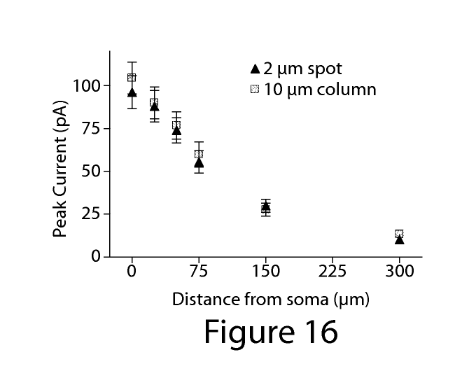

To determine the spatial precision with which L-Enk can signal in LC, we focused the photolysis beam down to a nominal spot of ˜2 μm in diameter and measured the current responses as the uncaging stimulus was applied at various distances along a straight line from the cell body. Due to strong scattering of UV photons by brain tissue, the effective illumination spot will be larger. Laser power was adjusted to elicit a response of ˜100 pA upon photolysis at the soma. Although care was taken to choose a trajectory that avoided major dendritic branches, due to the somewhat radial nature of the dendrites in the x-y plane, dendritic processes were typically present near the uncaging stimulus and most likely contributed to the measured responses. Nonetheless, the flash-evoked current amplitudes decreased with distance from the soma (FIG. 10A), yielding a half-maximal response at a distance of ˜100 μm. The activation kinetics of the evoked currents similarly decreased with distance from the soma (τₒₙ=0.31±0.01 s versus 0.89±0.14 s for photolysis at the soma and at a distance of 150 μm, respectively; FIG. 10B). Kinetic parameters could not be derived from the small individual responses to the uncaging stimulus at a distance of 300 μm and were thus excluded from this analysis. To determine if the observed profile depends on the shape of the uncaging stimulus (in this case a cone of light focused to a 2 μm diameter spot), we repeated this experiment using a collimated beam of 10 μm in diameter and adjusted the light intensity to again produce a response of ˜100 pA at the soma. As shown in the FIGURES, the spatial profiles for the elicited currents are super-imposable, suggesting that the spatial extent of signaling is a feature of enkephalin mobility in the LC and not a direct result of the optical configuration used for uncaging (FIG. 15, FIG. 16).

### Discussion of Measured Response Data

Neuropeptides are an important class of neurotransmitters that has received relatively little attention in comparison to other neuromodulators such as acetylcholine and the monoamines. Because it has been difficult to selectively stimulate neuropeptide release from distinct cell types (however see Ludwig, M. et al. (2006) Nat Rev Neurosci 7, 126-136), our understanding of neuropeptide signaling dynamics is limited. Photoactivatable molecules enable spatiotemporally precise delivery of endogenously occurring ligands in relatively intact brain tissue preparations. By creating photoactivatable opioid neuropeptides that are sufficiently inert to allow large responses to be generated with a brief uncaging stimulus, the caged L-Enk analogue CYLE provided robust, rapid and graded delivery of L-Enk in acute brain slices. The ability to spatially restrict release allowed us to selectively evoke currents from regions of neurons that can be effectively voltage clamped in order to accurately measure the reversal potential of the mu opioid receptor mediated K⁺ current, which was not previously possible in brain slices of LC. These features further enabled us to quantitatively characterize the mechanisms governing peptide clearance and delineate the limits of enkephalinergic volume transmission for the first time.

Based on extensive prior pharmacology, we identified the N-terminal Tyrosine side chain as a caging site where the relatively small CNB chromophore sufficiently attenuates potency on both L-Enk and Dyn-8. Peptides may be inherently more difficult to ‘cage’ than small molecules as the caging group will only interfere with one of multiple interaction sites with receptors. In particular, hydrophobic interactions contribute greatly to peptide-receptor binding and hydrophobic side chains lack functional handles for attaching caging groups. For these reasons the full length Dyn-17 or beta-endorphin may be more difficult to cage by the same approach.

CNB-tyrosine photolysis occurs with microseconds kinetics following a light flash (Sreekumar, R. et al. (1998) Methods Enzymol 291, 78-94; Tatsu, Y. et al. (1996) Biochemical and Biophysical Research Communications 227, 688-693), so the time-course of activation we observed in slices probably reflects the time required for ligand binding and engagement of the G-protein mediated signaling pathway that activates GIRK channels. Indeed, we observed a 50-100 ms delay from the flash to the current onset and a peak response within 1-2 seconds, which closely matches the rates observed for GABA_{B}R-mediated GIRK activation in dissociated cells using rapid perfusion techniques (Ingram, S. et al. (1997) Molecular Pharmacology 52, 136-143; Sodickson, D. L. et al. J Neurosci 16, 6374-6385). However, the offset kinetics we observed are orders of magnitude slower than those measured in dissociated cells, where currents cease within 1-2 s of agonist washout. Instead, photorelease produced deactivation kinetics that were only two-fold faster than those obtained with local perfusion, likely reflecting slow diffusion of released peptide away from the recorded cell in neural tissue and concomitant proteolytic cleavage. Indeed, addition of a protease inhibitor cocktail slowed deactivation of the response to photolysis over large areas.

Prompted by the ability to spatially confine L-Enk release, we revisited previous studies into opioid actions on rat LC neurons that were unable to unambiguously identify a K⁺ current using reversal potential measurements in brain slices (Osborne and Williams, 1996;). The reversal potential of the L-Enk-dependent current that we measured is accounted for by a pure K⁺ current and 80% of the outward current was blocked by a high concentration of Ba²⁺, consistent with a dominant role of GIRKs. It has been also been proposed that down-regulation of a cAMP-dependent standing Na⁺ current contributes 50% of the opioid response in rat LC (Alreja and Aghajanian, 1993; Alreja and Aghajanian, 1994). Although we cannot rule out that this component mediates the remaining 20% of the current not sensitive to Ba²⁺ or that this Na⁺ permeable channel may be enriched in the dendritic regions not activated by our soma-restricted uncaging stimulus, our results clearly demonstrate that the majority of the somatic current is carried by K⁺ channels and thus cannot reflect the closing of Na⁺ channels.

By varying the laser power and uncaging area used to photorelease L-Enk, we found correlation between the amplitude and duration of the outward current in voltage clamp and the duration of the pause in spontaneous firing recorded in current clamp (FIG. 7). The responses to the smallest stimuli demonstrate that small outwards currents near the detection limit are sufficient to prevent just a few action potentials without causing=significant hyperpolarization. At the opposite extreme, strong uncaging causes a large somatic hyperpolarization and pauses action potential firing for 30 s or longer. Thus, the effect of Enk on LC firing can be subtle or dramatic, highlighting that neuropeptides are capable of temporally precise actions in addition to volume transmission.

We found that L-Enk could generate opioid-receptor mediated currents when released ˜150 μm from the recorded cell. The slower onset kinetics observed when L-Enk was released at locations distant from the soma indicate that the photolyzed peptide diffused from the release site to activate receptors on the soma and proximal dendrites. These distances are large compared to those over which fast-acting neurotransmitters such as glutamate (Carter, A. G. et al. (2007) J Neurosci 27, 8967-8977) and GABA (Chalifoux, J. R. et al. (2011) J Neurosci 31, 4221-4232) can spread, as clearance mechanisms for these neurotransmitters are present at high density in neural tissue. Our results indicate that enkephalin can indeed function as a volume transmitter in LC and define the spatial profile of its spread from a single release site. Under the conditions of our experiments, L-Enk was essentially inactive when released 300 μm from the soma, which reflects the limit of detection by mu opioid receptors due to dilution of the peptide as it diffuses away from the release site. Assuming a diffusion-limited process, this absolute boundary depends not only on the initial quantity released, but also on the affinity of the receptor for the ligand. Our results may overestimate the mobility of L-Enk in LC due to activation of receptors on dendrites that are closer to the release site than the soma. The spatial limits of signaling may be different in other brain regions due to variations in the densities and identities of proteases and possible differences in diffusional mobility. Although we obtained similar results using two differently shaped photolysis beams, UV light does not provide good spatial control in the z-dimension. Thus, two-photon sensitive caged peptides will provide the most accurate means to measure these parameters in the future.

Here we have described novel photoactivatable tools for the study of opioid signaling within the mammalian brain. By caging both L-Enk and Dyn-8, we provide reagents that can be used to study mu, delta and kappa receptors. Using UV-mediated photolysis of caged L-Enk, we demonstrated that somatic mu receptors in the LC generate an outward current mediated primarily by K⁺ channels. These reagents allowed us to probe the mechanisms that regulate the spread of opioid signaling in brain tissue and revealed that with graded, temporally precise, and spatially confined release, neuropeptides are capable of subtle and relatively short-lasting but clearly defined modulation of neuronal function. This approach represents a general strategy for probing the spatiotemporal dynamics of neuropeptides and should be applicable to other peptide transmitters and useful to screen potential analogues and treatment preparations for neuronally mediated physiological responses and conditions of disease, pain or other etiology as described above and defined by the claims appended hereto.

1. A method of evaluating a neuronal activity or response to a neuropeptide, the method comprising
i) applying a caged peptide to a cell, the caged peptide comprising a peptide having an affinity for a receptor on a signaling cell and having a cage bound to the peptide and operative to prevent binding to the receptor, wherein the cage is selectively photodegradable by application of light
ii) applying light energy at an initial time t₀ and/or discrete region r₀ to free the peptide for interaction with the receptor, and
i) measuring a parameter or activity indicative of a the interaction.

2. The method of claim 1, wherein the caged peptide is a neuropeptide and the receptor is a μ, d or κ opioid, CCV, neurokinin, tachykinin or CCK receptor.

3. The method of claim 1, wherein the caged peptide is a neuropeptide selected from the group consisting of enkephalin, dynorphin, CCK or substance P.

4. The method of claim 1, wherein the parameter is a kinetic measurement of a K or Ca channel current or potential.

5. The method of claim 1, wherein the parameter is a time-resolved measurement of a downstream signaling activity.

6. The method of claim 1 wherein the light energy is applied locally and the parameter is measured locally over time in a synaptically-communicating region or tissue site following application of light to determine kinetics or an operative mechanism of one or more signaling pathways.

7. The method of claim 1 wherein the light energy is applied for a duration under about a millisecond, and the parameter is measured with high speed resolution to provide a response dataset to model effects of diffusion, uptake or release of an agonist or an antagonist.

8. The method of claim 7, wherein the caged peptide is applied to and/or the measurement of activity is performed on a group of one or only a few signaling processes.

9. The method of claim 8, wherein the measurement of signaling activity is an electrical potential.

10. The method of claim 8, wherein the caged peptide is applied to tissue, and the cage is controllably cleaved to release or activate the peptide at a defined site by selective illumination which may comprise scanning laser microscopy illumination.

11. The method of claim 10, wherein the measurement of signaling activity is performed by microscopic observation of a fluorescent reporter.

12. A method of providing a neuropeptide to a signaling receptor with high spatial and/or temporal resolution to enable measurement neuropeptide function or activity on a neuron, such method comprising:
caging the neuropeptide by providing a photocleavable side group effective to substantially prevent binding to a signaling receptor of a synaptic process;
applying the caged neuropeptide to a region of the synaptic process;
applying illumination at a time t₀ and position x₀ to cleave the side group and initiate binding to a signaling receptor at said position; and
locally detecting activity of the process.

13. The method of claim 12, applied to one or more candidate neuropeptides to evaluate relative effects, activity or interactions of the candidate neuropeptides.

14. The method of claim 12, wherein detecting activity includes one or more of
i) monitoring resting membrane properties of a cell
ii) measuring action potential initiation and propagation
iii) measuring intracellular calcium signaling; and
iv) measuring synaptic transmission; and
v) determining release, uptake, inhibition, interaction or other effect of a second peptide associated with the measured activity of the process.

15. A neuropeptide having a photocleavable caging group bound thereto, the caging group providing hindrance and effectively inhibiting binding of the neuropeptide to a receptor, such that the neuropeptide is selectively activated by illumination that cleaves the caging group to initiate interaction of the neuropeptide with the receptor.

16. The neuropeptide of claim 15, selected from the group consisting of a Leu- or Met-enkephalin, a dynorphin and a cholecystokinin.

17. The method of claim 12, applied to evaluate or quantify affinity and kinetics of binding and modulation of an agonist and/or antagonist of a possible modulator of the neuropeptide receptor.

18. The method of claim 1, wherein the light energy is applied to modulate neuronal activity in the brain or spinal cord to control pain, hunger, seizures, attention, cognition or other mental or corporeal function or condition.

19. The method of claim 18, wherein light energy is delivered by fiber optic stimulation of specific brain regions at specific times.

20. The method of claim 1, wherein the peptide is activated to create a local concentration of the active peptide effective for subsaturating activation or repression of high affinity receptors in the brain.

21. The method of claim 1, wherein the caged peptide is applied locally on or in the skin to control pain and/or inflammation, and light is delivered via UV lamp to activate the peptide.

22. The method of claim 1, wherein the caged peptide is activated at a synaptic head and the measurement of activity is performed at a distal synapse site.

23. A kit for performing the method of claim 1, comprising a caged neuropeptide for applying to tissue, and a reporter that provides a visible indication of the activity.

24. A small neuropeptide having a characteristic binding sequence or sub-sequence for interaction of the neuropeptide with a receptor to produce a synaptic response, wherein one or more positions of the sequence or sub-sequence are provided with a caging group to normally prevent interaction, the caging group having a photolysable bond such that photolysis activates the neuropeptide to initiate interaction thereby enabling identification or measurement of the response.

25. A kit comprising the caged neuropeptide of claim 24.

26. The kit of claim 25 further comprising instructions for use, the instructions including one or more of the following: indication of wavelength(s) for photolysis; identification of expected activity; calibration data for quantifying amount of active neuropeptide with amount of delivered light; specifications for preparing solutions for treating biological specimens with the neuropeptide; and specifications for assaying a synaptic response to the active neuropeptide.

27. A method of determining activity of a neuropeptide such method comprising the steps of
i) caging the neuropeptide with a caging group to suppress activity of the neuropeptide;
ii) applying the neuropeptide to a region including a cell; and
iii) irradiating at least a portion of the region to activate the caged neuropeptide so that it interacts with the cell to produce a response;
wherein the portion of the region is remote from the response.


SEQUENCE LISTING (4 sequences)

SEQ ID NO: 1 - protein, 5 aa, Artificial Sequence
  The sequence was designed and synthesized.
YGGFM

SEQ ID NO: 2 - protein, 5 aa, Artificial Sequence
  The sequence was designed and synthesized.
YGGFL

SEQ ID NO: 3 - protein, 11 aa, Artificial Sequence
  The sequence was designed and synthesized.
RPKPNNFFGLM

SEQ ID NO: 4 - protein, 6 aa, Artificial Sequence
  The sequence was designed and synthesized.
NFFGLM